(19) United States
(12) Patent Application Publication
(10) Pub. No.: US 2022/0096660 A1
(43) Pub. Date: Mar. 31, 2022

(54) METHODS FOR FORMING POLYPLEXES

(71) Applicant: Amryt Genetics Ltd, Dublin (IE)

(72) Inventor: Conall O'Broin, Dublin (IE)

(21) Appl. No.: 17/418,067

(22) PCT Filed: Dec. 30, 2019
(86) PCT No.: PCT/US2019/068954

§ 371 Date: Jun. 24, 2021

Related U.S. Application Data
(60) Provisional application No. 62/786,050, filed on Dec. 28, 2018.

(51) Int. Cl. A61K 48/00; A61K 38/39; A61K 47/64; A61K 47/26

(52) U.S. Cl. CPC A61K 48/0091; A61K 38/39; A61K 47/26; A61K 48/0041; A61K 47/6455; A61K 48/0066

(57) ABSTRACT

The present disclosure relates method for forming polyplexes, which find use in gene therapy applications as safe and non-toxic nucleic acid transfection agents.

### CROSS-REFERENCE TO RELATED APPLICATIONS

The present application claims the benefit of priority to U.S. Provisional Application No. 62/786,050, filed Dec. 28, 2018, the contents of which are hereby incorporated by reference in their entirety.

### BACKGROUND

Gene therapy is a rapidly growing area of nanomedicine for improving health conditions and correcting genetic disorders.^{[1]} Currently, the majority of gene delivery protocols utilize viral vectors as the gene carriers. However, concerns associated with viral vectors, including risks of triggering immunogenic responses and transgene insertional mutagenesis, limitations associated with large-scale production and low “cargo capacity” for genetic materials, along with the unpredictability of vector mobility remain unaddressed. ^{[2,3]}

Non-viral vectors offer a number of advantages over viral systems such as their potential for minimal immunogenicity, non-tumorigenicity, cost-effective manufacturing, high payload of nucleic acids and localized gene expression. Since 2010, the number of clinical trials for gene therapies using non-viral gene vectors has increased remarkably. Plasmid DNAs and small interfering RNAs (siRNA) have been formulated in at least 40 nanoparticle-based gene therapies for gene correction, therapeutic protein expression and antigen vaccination, with 12 major liposome systems investigated in 27 clinical trials and 7 polymer-based systems in 13 clinical trials.^{[3]} Among the polymer-based gene therapy clinical trials, the off-the-shelf cationic polymer polyethylenimine (PEI) has showed promise. However, PEI is nondegradable and severely hampered by safety concerns. ^{[4]} Therefore, tremendous effort has been made to improve the gene transfection efficiency and safety of polymeric gene vectors so that the polymer-based gene therapies can be brought closer to clinical applications. Several factors such as polymer size, molecular weight, degree of polymer branching, charge density as well as the composition of the formulation medium and the ratio of the polymer to the nucleic acid component influence the polyplexes' transfection efficiency and biological activity.^{[5]}

To be useful commercially, reproducible and scalable methods for the assembly of uniform and stable polyplex complexes with tight control of physical parameters are needed. In some embodiments, the present disclosure provides improved methods for preparing polyplexes, including methods that are scalable for commercial scale production.

### SUMMARY

In some embodiments, the present disclosure provides a method for making one or more polyplexes, the method comprising:

(a) providing:
i. a polymer in a first liquid stream;
ii. a nucleic acid component in a second liquid stream;
   (b) contacting the polymer in the first liquid stream with the nucleic acid component in the second liquid stream to form a polyplex having a size and charge that is suitable for therapeutic administration; and
   (c) isolating the polyplex to provide a stabilized polyplex.

In some embodiments, the method further comprises assessing and harvesting one or more polyplexes. In some embodiments, the method of the present invention is a continuous process whereby each polyplex formed is isolated from other polyplexes formed in the method. In some embodiments, each polyplex is separated from other polyplexes during a stabilization phase after contacting the first and second liquid streams.

In some embodiments, the method further comprises filtering, washing, freezing and/or lyophilizing the stabilized polyplexes.

### BRIEF DESCRIPTION OF THE DRAWINGS

FIG. 1 describes an exemplary method of the present disclosure for manufacturing polyplexes.

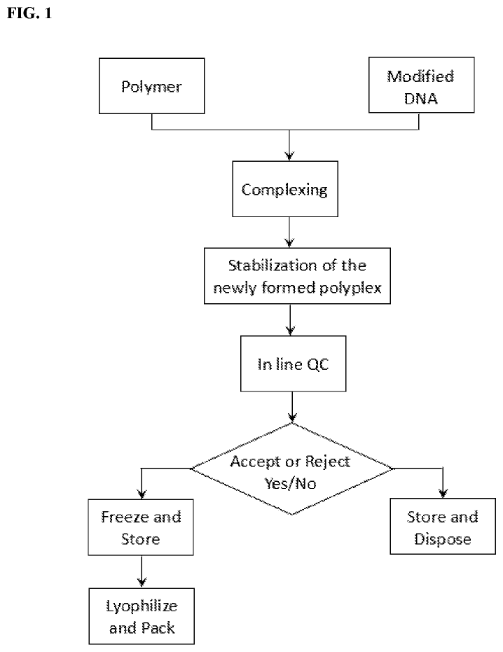

### DETAILED DESCRIPTION

As used above, and throughout this disclosure, the following terms, unless otherwise indicated, shall be understood to have the following meanings. If a term is missing, the conventional term as known to one skilled in the art controls.

As used herein, the terms “including,” “containing,” and “comprising” are used in their open, non-limiting sense.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “about” when immediately preceding a numerical value means a range (e.g., plus or minus 10% of that value). For example, “about50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range. Concentrations that are given as percentages refer to mass ratios, unless indicated differently.

The term “disorder” is used in this disclosure to mean, and is used interchangeably with, the terms disease, condition, or illness, unless otherwise indicated.

By using the terms “pharmaceutically acceptable” or “pharmacologically acceptable” it is intended to mean a material which is not biologically, or otherwise, undesirable—the material may be administered to an individual without causing any substantially undesirable biological effects or interacting in a deleterious manner with any of the components of the composition in which it is contained.

All documents referenced herein are incorporated by reference in their entireties for all purposes.

As used herein, “alkyl” means a straight chain or branched saturated chain having from 1 to 40 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl and the like, and longer alkyl groups, such as heptyl, and octyl and the like. An alkyl group can be unsubstituted or substituted. Alkyl groups containing three or more carbon atoms may be straight, or branched. As used herein, “lower alkyl” means an alkyl having from 1 to 10 carbon atoms.

As used herein, an “alkenyl” includes an unbranched or branched hydrocarbon chain containing 2-40 carbon atoms. The “alkenyl” group contains at least one double bond. The double bond of an alkenyl group can be unconjugated or conjugated to another unsaturated group. Examples of alkenyl groups include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. An alkenyl group can be unsubstituted or substituted. Alkenyl, as defined herein, may also be branched or straight.

As used herein, “alkynyl” includes an unbranched or branched unsaturated hydrocarbon chain containing 2-40 carbon atoms. The “alkynyl” group contains at least one triple bond. The triple bond of an alkynyl group can be unconjugated or conjugated to another unsaturated group. Examples of alkynyl groups include, but are not limited to, ethynyl, propynyl, butynyl, pentynyl, hexynyl, methylpropynyl, 4-methyl-1-butynyl, 4-propyl-2-pentynyl, 4-butyl-2-hexynyl and the like. An alkynyl group can be unsubstituted or substituted.

It should also be noted that any carbon as well as heteroatom with unsatisfied valences described herein is assumed to have the sufficient number of hydrogen atom(s) to satisfy the valences.

The term “halo” or “halogen” refers to fluorine, chlorine, bromine, or iodine.

The term “haloalkyl” as used herein refers to an alkyl group, as defined herein, which is substituted by one or more halogen. Examples of haloalkyl groups include, but are not limited to, trifluoromethyl, difluoromethyl, pentafluoroethyl, trichloromethyl, etc.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted. Furthermore, when containing two fused rings the aryl groups herein defined may have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 18 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. The substituents can themselves be optionally substituted. Examples include, but are not limited to, benzothiophene, furyl, thienyl, pyrrolyl, pyridyl, pyrazinyl, pyrazolyl, pyridazinyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, benzoimidazolyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two fused rings the heteroaryl groups herein defined may have an unsaturated or partially saturated ring fused with a fully saturated ring.

As used herein, the term “cycloalkyl” refers to a saturated or partially saturated, monocyclic, fused or spiro polycyclic, carbocycle having from 3 to 18 carbon atoms per ring. The cycloalkyl ring or carbocycle may be optionally substituted by one or more substituents, e.g., 1 to 5 substituents, at any point of attachment. The substituents can themselves be optionally substituted. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, and derivatives thereof.

As used herein, the term “cycloalkenyl” refers to a partially saturated, monocyclic, fused or spiro polycyclic, carbocycle having from 3 to 18 carbon atoms per ring and contains at least one double bond. The cycloalkenyl ring may be optionally substituted by one or more substituents, e.g., 1 to 5 substituents, at any point of attachment. The substituents can themselves be optionally substituted.

As used herein, the term “heterocycloalkyl” or “heterocyclyl” refers to a saturated or partially unsaturated and non-aromatic monocyclic, or fused or spiro, polycyclic, ring structure of 4- to- 18 atoms containing carbon and heteroatoms taken from oxygen, nitrogen, or sulfur and wherein there is not delocalized it-electrons (aromaticity) shared among the ring carbon or heteroatoms. The heterocycloalkyl or heterocyclyl ring structure may be substituted by one or more substituents. The substituents can themselves be optionally substituted. Examples of heterocycloalkyl or heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, homotropanyl, dihydrothiophen-2(3H)-onyl, tetrahydrothiophene 1,1-dioxide, 2,5-dihydro-1H-pyrrolyl, imidazolidin-2-one, pyrrolidin-2-one, dihydrofuran-2(3H)-one, 1,3-dioxolan-2-one, isothiazolidine 1,1-dioxide, 4,5-dihydro-1H-imidazolyl, 4,5-dihydrooxazolyl, oxiranyl, pyrazolidinyl, 4H-1,4-thiazinyl, thiomorpholinyl, 1,2,3,4-tetrahydropyridinyl, 1,2,3,4-tetrahydropyrazinyl, 1,3-oxazinan-2-one, tetrahydro-2H-thiopyran 1,1-dioxide, 7-oxabicyclo[2.2.1]heptanyl, 1,2-thiazepane 1,1-dioxide, octahydro-2H-quinolizinyl, 1,3-diazabicyclo[2.2.2]octanyl, 2,3-dihydrobenzo[b][1,4]dioxine, 3-azabicyclo[3.2.1]octanyl, 8-azaspiro[4.5]decane, 8-oxa-3-azabicyclo[3.2.1]octanyl, 2-azabicyclo[2.2.1]heptane, 2,8-diazaspiro[5.5]undecanyl, 2-azaspiro[5.5]undecanyl, 3-azaspiro[5.5]undecanyl, decahydroisoquinolinyl, 1-oxa-8-azaspiro[4.5]decanyl, 8-azabicyclo[3.2.1]octanyl, 1,4′-bipiperidinyl, azepanyl, 8-oxa-3-azabicyclo[3.2.1]octanyl, 3,4-dihydro-2H-benzo[b][1,4]oxazinyl, 5,6,7,8-tetrahydroimidazo[1,2-a]pyridinyl, 1,4-diazepanyl, phenoxathiinyl, benzo[d][1,3]dioxolyl, 2,3-dihydrobenzofuranyl, 2,3-dihydrobenzo[b][1,4] dioxinyl, 4-(piperidin-4-yl)morpholinyl, 3-azaspiro[5.5]undecanyl, decahydroquinolinyl, piperazin-2-one, 1-(pyrrolidin-2-ylmethyl)pyrrolidinyl, 1,3′-bipyrrolidinyl, and 6,7,8,9-tetrahydro-1H,5H-pyrazolo[1,2-a][1,2]diazepinyl.

Numerical ranges, as used herein, are intended to include sequential integers, unless otherwise noted. For example, a range expressed as “from 0 to 5” would include 0, 1, 2, 3, 4 and 5.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. If not specified otherwise, suitable substituents used in the optional substitution of the described groups include, without limitation, oxo, -halogen, C₁-C₆ alkyl, C₁-C₆ alkoxy, C₁-C₆ haloalkyl, C₁-C₆ haloalkoxy, —OC₁-C₆ alkenyl, —OC₁-C₆ alkynyl, —C₁-C₆ alkenyl, —C₁-C₆ alkynyl, —OH, CN (cyano), —CH₂CN, —OP(O)(OH)₂, —C(O)OH, —OC(O)C₁-C₆ alkyl, —C(O)C₁-C₆ alkyl, —C(O)—C₀-C₆ alkylenyl-cycloalkyl, —C(O)—C₀-C₆ alkylenyl-heterocycloalkyl, —C(O)—C₀-C₆ alkylenyl-aryl, —C(O)—C₀-C₆ alkylenyl-heteroaryl, —OC(O)OC₁-C₆ alkyl, NH₂, NH(C₁-C₆ alkyl), N(C₁-C₆ alkyl)₂, —C(O)NH₂, —C(O)NH(C₁-C₆ alkyl), —C(O)N(C₁-C₆ alkyl)₂, —C(O)NH cycloalkyl, —C(O)N(C₁-C₆ alkyl)cycloalkyl, —C(O)NHheterocycloalkyl, —C(O)N(C₁-C₆ alkyl)heterocycloalkyl, —C(O)NHaryl, —C(O)N(C₁-C₆ alkyl)aryl, —C(O)NHheteroaryl, —C(O)N(C₁-C₆ alkyl)heteroaryl, —S(O)₂—C₁-C₆ alkyl, —S(O)₂—C₁-C₆ haloalkyl, —S(O)₂-cycloalkyl, —S(O)₂-heterocycloalkyl, —S(O)₂-aryl, —S(O)₂-heteroaryl-C₀-C₆ alkylenyl-S(O)₂NH₂, —S(O)₂NHC₁-C₆ alkyl, —S(O)₂N(C₁-C₆ alkyl)₂, —S(O)₂NHcycloalkyl, —S(O)₂NHheterocycloalkyl, —S(O)₂NHaryl, —S(O)₂NHhetereoaryl, —NHS(O)₂C₁-C₆ alkyl, —N(C₁-C₆ alkyl)S(O)₂(C₁-C₆ alkyl), —NHS(O)₂aryl, —N(C₁-C₆ alkyl)S(O)₂ aryl, —NHS(O)₂ heteroaryl, —N(C₁-C₆ alkyl)S(O)₂ heteroaryl, —NHS(O)₂ cycloalkyl, —N(C₁-C₆ alkyl)S(O)₂ cycloalkyl, —NHS(O)₂ heterocycloalkyl, —N(C₁-C₆ alkyl)S(O)₂ heterocycloalkyl, —N(C₁-C₆ alkyl)S(O)₂ aryl, —C₀-C₆ alkylenyl-aryl, —C₀-C₆ alkylenyl-heteroaryl, —C₀-C₆ alkylenyl-cycloalkyl, —C₀-C₆ alkylenyl-heterocycloalkyl, —O-aryl, —NH-aryl, and N(C₁-C₆ alkyl)aryl. The substituents can themselves be optionally substituted. When a multifunctional moiety is shown, the point of attachment to the core is indicated by a line, e.g., (cycloalkyloxy)alkyl-refers to alkyl being the point of attachment to the core while cycloalkyl is attached to alkyl via the oxy group. “Optionally substituted” also refers to “substituted” or “unsubstituted”, with the meanings described above.

As used herein, the term “linker” or “linking moiety” refers to a group that connects two groups and has a backbone of between 0 and 100 atoms. A linker or linkage may be a covalent bond (i.e., backbone of 0 atoms) that connects two groups or a chain of between 1 and 100 atoms in length, for example of about 1, 2, 3, 4, 5, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 carbon atoms in length, where the linker may be linear, branched, cyclic or a single atom. In certain cases, one or more carbon atoms of a linker backbone may be optionally substituted with a sulfur, nitrogen or oxygen heteroatom. The bonds between backbone atoms may be saturated or unsaturated. The linker may include one or more substituent groups, for example an alkyl, aryl or alkenyl group. A linker may include, without limitations, oligo(ethylene glycol), ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

Unless otherwise stated, the term molecular weight refers to weight average molecular weight (Mw).

The term “heteroalkylene” refers to a divalent alkylene having one or more carbon atoms replaced with a sulfur, oxygen, or NRd where Rd is hydrogen or alkyl. The heteroalkylene can be linear, branched, cyclic, or combinations thereof.

The term “heteroalkenylene” refers to divalent straight or branched chain hydrocarbyl groups having at least one carbon-carbon double bond, and one or more heteroatoms (e.g., N, S or O) in the backbone thereof.

The term “heteroalkynylene” refers to divalent straight or branched chain hydrocarbyl groups having at least one carbon-carbon triple bond, and one or more heteroatoms (e.g., N, S or O) in the backbone thereof.

The term “polyplex” as used herein refers to a complex between a nucleic acid and a polymer. The nucleic acid is bound, encapsulated or linked to the polymer via non-covalent forces, for example, electrostatic interactions.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid.

The term “nanoplasmid” refers to a circular DNA sequence having a reduced bacterial sequence which provides a smaller plasmid with the desired gene insert. For example, nanoplasmids produced by an antibiotic free RNA-OUT selection system and methods of making the same are described in U.S. Pat. No. 9,109,012, which are hereby incorporated by reference in their entirety patented by Nature Technology.

The term “nucleic acid” refers to a biological polymer of nucleotide bases, and may include but is not limited to deoxyribonucleic acid (DNA), ribonucleic acid (RNA), micro RNA (miRNA), and peptide nucleic acid (PNA).

The term “minicircle” refers to small, minimally sized circular DNA derived from a parental plasmid by intramolecular recombination to remove bacterial replication sequences.

The term “gene editing system” refers to a system capable of altering a target nucleic acid by one of many DNA repair pathways.

The term “assessing” is used in this disclosure to mean evaluating one or more characteristics of the polyplex to determine if the polyplex conforms to specification, including but not limited to the size, shape, zeta potential, form (e.g. non-agglomerated form), or refractive index.

### Methods

The present disclosure provides methods for manufacturing polyplexes where the methods involve mixing a polymer and a nucleic acid component (such as DNA) to provide a stabilized polyplex that is isolated.

Conventional processes for preparing polyplexes provide for mixing a solution of a suitable polymer with a solution of a suitable DNA (or RNA). The resulting mixture forms complexes between the DNA (or RNA) and polymer, generally via an electrostatic interaction. The resulting polyplexes are present in a range of sizes, and include agglomerated polyplexes that are unsuitable for absorption into the targeted cells. The methods described herein provide for controlled formation of such polyplexes by contacting selected volumes and concentrations of DNA and polymer solutions, under controlled conditions, such that the resulting polyplexes have a relatively narrow and well-defined distribution of polyplex characteristics (e.g., size, charge, etc.). Further, since the polyplexes are formed sequentially, automated quality control methods can be used to discard polyplexes having characteristics outside of the defined manufacturing specifications, and/or select and isolate polyplexes meeting the defined manufacturing specifications. In this manner, the resulting polyplexes have more uniform characteristics and improved therapeutic effectiveness.

In some embodiments, the present disclosure provides a method for making one or more polyplexes, the method comprising:

(a) providing:
i. a polymer in a first liquid stream;
ii. a nucleic acid component in a second liquid stream;
   (b) contacting the polymer in the first liquid stream with the nucleic acid component in the second liquid stream to form a polyplex having a size and charge that is suitable for therapeutic administration (such as transdermal administration); and
   (c) isolating the polyplex to provide a stabilized polyplex.

In some embodiments, the first liquid stream and second liquid streams are solutions.

In some embodiments, the present disclosure provides a continuous method for making polyplexes.

In some embodiments of the methods of the present disclosure, the polymer in the first liquid stream with the nucleic acid component in the second liquid stream are contacted in an amount that provides a specific ratio of nucleic acid component to polymer, as described herein.

In some embodiments, the method comprises flowing the first liquid stream and the second liquid stream through a flow-regulating device at a rate that provides a polyplex having a size and charge that is suitable for transdermal administration or injection.

In some embodiments, the method comprises flowing the first liquid stream and the second liquid stream through a flow-regulating device at a microliter rate.

In some embodiments, the polymer-containing first liquid stream and nucleic acid-containing second liquid stream each independently flow through a flow-regulating device at a rate from about 1 μL/min to about 1000 μL/min, including about 1.0 μL/min, about 2.0 μL/min, about 3.0 μL/min, about 4.0 μL/min, about 5.0 μL/min, about 6.0 μL/min, about 7.0 μL/min, about 8.0 μL/min, about 9.0 μL/min, about 10 μL/min, about 15 μL/min, about 20 μL/min, about 25 μL/min, about 30 μL/min, about 35 μL/min, about 40 μL/min, about 45 μL/min, about 50 μL/min, about 55 μL/min, about 60 μL/min, about 65 μL/min, about 70 μL/min, about 75 μL/min, about 80 μL/min, about 85 μL/min, about 90 μL/min, about 95 μL/min, about 100 μL/min, about 150 μL/min, about 200 μL/min, about 250 μL/min, about 300 μL/min, about 350 μL/min, about 400 μL/min, about 450 μL/min, about 500 μL/min, about 550 μL/min, about 600 μL/min, about 650 μL/min, about 700 μL/min, about 750 μL/min, about 800 μL/min, about 850 μL/min, about 900 μL/min, about 950 μL/min, and about 1000 μL/min, including all ranges there between.

In some embodiments, the polymer-containing first liquid stream and nucleic acid-containing second liquid stream each independently flow through a flow-regulating device at a rate of about 1 μL/min, about 2.0 μL/min, about 3.0 μL/min, about 4.0 μL/min, about 5.0 μL/min, about 6.0 μL/min, about 7.0 μL/min, about 8.0 μL/min, about 9.0 μL/min, about 10 μL/min, about 15 μL/min, about 20 μL/min, about 25 μL/min, about 30 μL/min, about 35 μL/min, about 40 μL/min, about 45 μL/min, about 50 μL/min, about 55 μL/min, about 60 μL/min, about 65 μL/min, about 70 μL/min, about 75 μL/min, about 80 μL/min, about 85 μL/min, about 90 μL/min, about 95 μL/min, about 100 μL/min, about 150 μL/min, about 200 μL/min, about 250 μL/min, about 300 μL/min, about 350 μL/min, about 400 μL/min, about 450 μL/min, about 500 μL/min, about 550 μL/min, about 600 μL/min, about 650 μL/min, about 700 μL/min, about 750 μL/min, about 800 μL/min, about 850 μL/min, about 900 μL/min, about 950 μL/min, or about 1000 μL/min.

In some embodiments, the method is conducted at a pressure of about 500 mbar to about 2 bar, including about 500 mbar, about 550 mbar, about 600 mbar, about 650 mbar, about 700 mbar, about 750 mbar, about 800 mbar, about 850 mbar, about 900 mbar, about 950 mbar, about 1 bar, about 1.1 bar, about 1.2 bar, about 1.3 bar, about 1.4, about 1.5 bar, about 1.6 bar, about 1.7 bar, about 1.8 bar, about 1.9 bar, and about 2 bar, including all ranges there between. In some embodiments, the method is conducted at a pressure of about 500 mbar, about 550 mbar, about 600 mbar, about 650 mbar, about 700 mbar, about 750 mbar, about 800 mbar, about 850 mbar, about 900 mbar, about 950 mbar, about 1 bar, about 1.1 bar, about 1.2 bar, about 1.3 bar, about 1.4, about 1.5 bar, about 1.6 bar, about 1.7 bar, about 1.8 bar, about 1.9 bar, or about 2 bar. In some embodiments, the method is conducted at a pressure of about 1 to about 2 bars. All pressures described herein are absolute pressures.

In some embodiments, the first and second liquid streams conveying the polymer and the nucleic acid component are enclosed in channels having a width of about 1 mm to about 12 mm, including about 1 mm, about 2 mm, about 3 mm, about 4 mm, about 5 mm, about 6 mm, about 7 mm, about 8 mm, about 9 mm, about 10 mm, about 11 mm, and about 12 mm, including all ranges there between. In some embodiments, the width of the channels are about 1 mm, about 2 mm, about 3 mm, about 4 mm, about 5 mm, about 6 mm, about 7 mm, about 8 mm, about 9 mm, about 10 mm, about 11 mm, or about 12 mm.

In some embodiments, the first and second liquid streams conveying the polymer and the nucleic acid component are enclosed in channels having a height of about 1 mm to about 12 mm, including about 1 mm, about 2 mm, about 3 mm, about 4 mm, about 5 mm, about 6 mm, about 7 mm, about 8 mm, about 9 mm, about 10 mm, about 11 mm, and about 12 mm, including all ranges there between. In some embodiments, the height of the channels are about 1 mm, about 2 mm, about 3 mm, about 4 mm, about 5 mm, about 6 mm, about 7 mm, about 8 mm, about 9 mm, about 10 mm, about 11 mm, or about 12 mm. The cross section of the channel can be approximately rectangular, oval, or circular.

In some embodiments, the flow-regulating device is selected from the group consisting of a positive displacement pump, a syringe driven pump, a pressure driven pump, and a gravity feed pump or system.

In some embodiments, the contacting step uses a nozzle, a micro fluidics mixing device, a touch tube, a liquid bridge, a vertical mixer, a rotating double tube, or an atomizer.

The following embodiments provide non-limiting examples of methods for contacting the polymer-containing first liquid stream and nucleic acid-containing second liquid stream.

Nozzles. In some embodiments, nozzles are used in the methods of the present disclosure. In some embodiments, one nozzle injects a controlled amount of nucleic acid component into an oil/liquid channel and, at an appointed time and position that are determined by sensors (such as cameras) by means of a control system, a second nozzle injects the polymer-containing first liquid stream into the nucleic acid-containing second liquid stream to provide a polyplex. This precision of delivering multiple injections under precisely controlled time, velocity, volume, density and position parameters provides for controlled formation of polyplexes with relatively uniform and controlled properties. In some embodiments, two nozzles (one for the nucleic acid-containing second liquid stream and one for the polymer-containing first liquid stream) are oppositely configured to inject the two opposing liquid streams at high velocity into a mixing chamber (i.e., impinging jet mixing).

Microfluidics. In some embodiments, microfluidics are used in the methods of the present disclosure. In some embodiments, two microfluidic streams (e.g. the polymer-containing first liquid stream and nucleic acid-containing second liquid stream) come together to form polyplexes. In some embodiments, pulses of liquid containing controlled amounts of nucleic acid component and polymer (e.g., HPAE) travel along each microfluidic stream to discharge the correct amount of raw material (e.g., nucleic acid component or HPAE) from each microfluidic stream into the mixing area. Mixing is instantaneous and precise.

Touch Tubes. In some embodiments, touch tubes are used in the methods of the present disclosure. In some embodiments, the polymer-containing first liquid stream and nucleic acid-containing second liquid stream come together to meet and mix in a touch tube after they have been discharged from their respective nozzles or microfluidic streams. In some embodiments, the tubes face each other, separated by a small air gap. In some embodiments, multiple touch tubes individually or collectively discharge or the polymer-containing first liquid stream and nucleic acid-containing second liquid stream into carrier streams.

Liquid Bridging: In some embodiments, liquid bridging is used in the methods of the present disclosure. In some embodiments, the polymer-containing first liquid stream and nucleic acid-containing second liquid stream are conveyed through generally parallel tubes onto a vertically oriented platform where they meet and complex. The resultant polyplex can then slide off the vertical platform into a liquid channel as described herein, which conveys the formed polyplexes to e.g., a reservoir for harvesting.

Vertical mixer: In some embodiments, vertical mixing is used in the methods of the present disclosure. In some embodiments, the polymer-containing first liquid stream and nucleic acid-containing second liquid stream are separately fed in two concentrically oriented tubes, one tube inside the other. Upon discharge, the contents of the inner tube (e.g., nucleic acid component or polymer) mixes with the contents of the concentrically oriented outer tube (e.g., nucleic acid component or polymer) to form the polyplex, Once the polyplex forms, the density changes, and the formed polyplexes move outwards and flow into a liquid channel, as described herein, which conveys the formed polyplexes to, e.g., a reservoir for harvesting.

Rotating Vertical Mixer: In some embodiments, a rotating vertical mixer is used in the methods of the present disclosure. In some embodiments, the polymer-containing first liquid stream and nucleic acid-containing second liquid stream are fed up a tube in tube scenario and the centrifugal forces cause both materials to move out of the center at different rates thereby mixing. Once the polyplex forms, the density changes, whereby the formed polyplexes move outwards and flows into a liquid channel, as described herein, which conveys the formed polyplexes to, e.g., a reservoir for harvesting.

In some embodiments, the contacting step uses a nozzle. In some embodiments, the nozzles are directed to provide a pulsed flow contact between the first liquid stream and the second liquid stream made at 90° to the direction of travel of each individual material The angle of the nozzles relative to the liquid transport channel can vary from 0° to 90°.

In some embodiments, the contacting step occurs in an air gap or in the carrier oil within a liquid channel as described herein. In some embodiments, polyplex formation begins instantly without external mixing (such as mechanical mixing). In some embodiments, polyplex formation requires external mixing after the contacting step. In some embodiments, the external mixing comprises blowing air streams over the mixture after the contacting step. In some embodiments, the air stream assists the movement of the newly formed polyplexes during the stabilizing phase.

In some embodiments, the volumes of the polymer-containing first liquid stream and nucleic acid-containing second liquid stream in the contacting step are in the microliter range (e.g., about 1 μL to about 1000 μL). In some embodiments, the volumes of the polymer-containing first liquid stream and nucleic acid-containing second liquid stream in the contacting step are less than about 5 μL.

In some embodiments, the volume of the first liquid stream and the second liquid stream is each independently from about 1 μL to about 1000 μL, including about 1 μL, about 5 μL, about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, about 100 μL, about 105 μL, about 110 μL, about 115 μL, about 120 μL, about 125 μL, about 130 μL, about 135 μL, about 140 μL, about 145 μL, about 150 μL, about 155 μL, about 160 μL, about 165 μL, about 170 μL, about 175 μL, about 180 μL, about 185 μL, about 190 μL, about 195 μL, about 200 μL, about 205 μL, about 210 μL, about 215 μL, about 220 μL, about 225 μL, about 230 μL, about 235 μL, about 240 μL, about 245 μL, about 250 μL, about 255 μL, about 260 μL, about 265 μL, about 270 μL, about 275 μL, about 280 μL, about 285 μL, about 290 μL, about 295 μL, about 300 about 305 μL, about 310 μL, about 315 μL, about 320 μL, about 325 μL, about 330 μL, about 335 μL, about 340 μL, about 345 μL, about 350 μL, about 355 μL, about 360 μL, about 365 μL, about 370 μL, about 375 μL, about 380 μL, about 385 μL, about 390 μL, about 395 μL, about 400 μL, about 405 μL, about 410 μL, about 415 μL, about 420 μL, about 425 μL, about 430 μL, about 435 μL, about 440 μL, about 445 μL, about 450 μL, about 455 μL, about 460 μL, about 465 μL, about 470 μL, about 475 μL, about 480 μL, about 485 μL, about 490 μL, about 495 μL, about 500 μL, about 505 μL, about 510 μL, about 515 μL, about 520 μL, about 525 μL, about 530 μL, about 535 μL, about 540 μL, about 545 μL, about 550 μL, about 555 μL, about 560 μL, about 565 μL, about 570 μL, about 575 μL, about 580 μL, about 585 μL, about 590 μL, about 595 μL, about 600 μL, about 605 μL, about 610 μL, about 615 μL, about 620 μL, about 625 μL, about 630 μL, about 635 μL, about 640 μL, about 645 μL, about 650 μL, about 655 μL, about 660 μL, about 665 μL, about 670 μL, about 675 μL, about 680 μL, about 685 μL, about 690 μL, about 695 μL, about 700 μL, about 705 μL, about 710 μL, about 715 μL, about 720 μL, about 725 μL, about 730 μL, about 735 μL, about 740 μL, about 745 μL, about 750 μL, about 755 μL, about 760 μL, about 765 μL, about 770 μL, about 775 μL, about 780 μL, about 785 μL, about 790 μL, about 795 μL, about 800 μL, about 810 μL, about 815 μL, about 820 μL, about 825 μL, about 830 μL, about 835 μL, about 840 μL, about 845 μL, about 850 μL, about 855 μL, about 860 μL, about 865 μL, about 870 μL, about 875 μL, about 880 μL, about 885 μL, about 890 μL, about 895 μL, about 900 μL, about 905 μL, about 910 μL, about 915 μL, about 920 μL, about 925 μL, about 930 μL, about 935 μL, about 940 μL, about 945 μL, about 950 μL, about 955 μL, about 960 μL, about 965 μL, about 970 μL, about 975 μL, about 980 μL, about 985 μL, about 990 μL, about 995 μL, and about 1000 μL, including all ranges there between.

In some embodiments, the volume of the first liquid stream and the second liquid stream is each independently about 1 μL, about 5 μL, about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, about 100 μL, about 105 μL, about 110 μL, about 115 μL, about 120 μL, about 125 μL, about 130 μL, about 135 μL, about 140 μL, about 145 μL, about 150 μL, about 155 μL, about 160 μL, about 165 μL, about 170 μL, about 175 μL, about 180 μL, about 185 μL, about 190 μL, about 195 μL, about 200 μL, about 205 μL, about 210 μL, about 215 μL, about 220 μL, about 225 μL, about 230 μL, about 235 μL, about 240 μL, about 245 μL, about 250 μL, about 255 μL, about 260 μL, about 265 μL, about 270 μL, about 275 μL, about 280 μL, about 285 μL, about 290 μL, about 295 μL, about 300 about 305 μL, about 310 μL, about 315 μL, about 320 μL, about 325 μL, about 330 μL, about 335 μL, about 340 μL, about 345 μL, about 350 μL, about 355 μL, about 360 μL, about 365 μL, about 370 μL, about 375 μL, about 380 μL, about 385 μL, about 390 μL, about 395 μL, about 400 μL, about 405 μL, about 410 μL, about 415 μL, about 420 μL, about 425 μL, about 430 μL, about 435 μL, about 440 μL, about 445 μL, about 450 μL, about 455 μL, about 460 μL, about 465 μL, about 470 μL, about 475 μL, about 480 μL, about 485 μL, about 490 μL, about 495 μL, about 500 μL, about 505 μL, about 510 μL, about 515 μL, about 520 μL, about 525 μL, about 530 μL, about 535 μL, about 540 μL, about 545 μL, about 550 μL, about 555 μL, about 560 μL, about 565 μL, about 570 μL, about 575 μL, about 580 μL, about 585 μL, about 590 μL, about 595 μL, about 600 μL, about 605 μL, about 610 μL, about 615 μL, about 620 μL, about 625 μL, about 630 μL, about 635 μL, about 640 μL, about 645 μL, about 650 μL, about 655 μL, about 660 μL, about 665 μL, about 670 μL, about 675 μL, about 680 μL, about 685 μL, about 690 μL, about 695 μL, about 700 μL, about 705 μL, about 710 μL, about 715 μL, about 720 μL, about 725 μL, about 730 μL, about 735 μL, about 740 μL, about 745 μL, about 750 μL, about 755 μL, about 760 μL, about 765 μL, about 770 μL, about 775 μL, about 780 μL, about 785 μL, about 790 μL, about 795 μL, about 800 μL, about 810 μL, about 815 μL, about 820 μL, about 825 μL, about 830 μL, about 835 μL, about 840 μL, about 845 μL, about 850 μL, about 855 μL, about 860 μL, about 865 μL, about 870 μL, about 875 μL, about 880 μL, about 885 μL, about 890 μL, about 895 μL, about 900 μL, about 905 μL, about 910 μL, about 915 μL, about 920 μL, about 925 μL, about 930 μL, about 935 μL, about 940 μL, about 945 μL, about 950 μL, about 955 μL, about 960 μL, about 965 μL, about 970 μL, about 975 μL, about 980 μL, about 985 μL, about 990 μL, about 995 μL, or about 1000 μL.

In some embodiments, the volume of the first liquid stream and the second liquid stream is each independently a volume of about 1 μL to about 300 μL. In some embodiments, the volume of the first liquid stream and the second liquid stream is each independently about 10 μL.

In some embodiments, the volume of the first liquid stream and the second liquid stream (e.g., each pulse of the first and second liquid streams, for example from a nozzle) is each independently about 0.1 μL, about 0.5 μL, about 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 51 μL, about 52 μL, about 53 μL, about 54 μL, about 55 μL, about 56 μL, about 57 μL, about 58 μL, about 59 μL, about 60 μL, about 61 μL, about 62 μL, about 63 μL, about 64 μL, about 65 μL, about 67 μL, about 68 μL, about 69 μL, about 70 μL, about 71 μL, about 72 μL, about 73 μL, about 74 μL, about 75 μL, about 76 μL, about 77 μL, about 78 μL, about 79 μL, about 80 μL, about 81 μL, about 82 μL, about 83 μL, about 84 μL, about 85 μL, about 86 μL, about 87 μL, about 88 μL, about 89 μL, about 90 μL, about 91 μL, about 92 μL, about 93 μL, about 94 μL, about 95 μL, about 96 μL, about 97 μL, about 98 μL, about 99 μL, about 100 μL, about 101 μL, about 102 μL, about 103 μL, about 104 μL, about 105 μL, about 106 μL, about 107 μL, about 108 μL, about 109 μL, about 110 μL, about 111 μL, about 112 μL, about 113 μL, about 114 μL, about 115 μL, about 116 μL, about 117 μL, about 118 μL, about 119 μL, about 120 μL, about 121 μL, about 122 μL, about 123 μL, about 124 μL, about 125 μL, about 126 μL, about 127 μL, about 128 μL, about 129 μL, about 130 μL, about 131 μL, about 132 μL, about 133 μL, about 134 μL, about 135 μL, about 136 μL, about 137 μL, about 138 μL, about 139 μL, about 140 μL, about 141 μL, about 142 μL, about 143 μL, about 144 μL, about 145 μL, about 146 μL, about 147 μL, about 148 μL, about 149 μL, about 150 μL, about 151 μL, about 152 μL, about 153 μL, about 154 μL, about 155 μL, about 156 μL, about 157 μL, about 158 μL, about 159 μL, about 160 μL, about 161 μL, about 162 μL, about 163 μL, about 164 μL, about 165 μL, about 166 μL, about 167 μL, about 168 μL, about 169 μL, about 170 μL, about 171 μL, about 172 μL, about 173 μL, about 174 μL, about 175 μL, about 176 μL, about 177 μL, about 178 μL, about 179 μL, about 180 μL, about 181 μL, about 182 μL, about 183 μL, about 184 μL, about 185 μL, about 186 μL, about 187 μL, about 188 μL, about 189 μL, about 190 μL, about 191 μL, about 192 μL, about 193 μL, about 194 μL, about 195 μL, about 196 μL, about 197 μL, about 198 μL, about 199 μL, about 200, about 201 μL, about 202 μL, about 203 μL, about 204 μL, about 205 μL, about 206 μL, about 207 μL, about 208 μL, about 209 μL, about 210 μL, about 211 μL, about 212 μL, about 213 μL, about 214 μL, about 215 μL, about 216 μL, about 217 μL, about 218 μL, about 219 μL, about 220 μL, about 221 μL, about 222 μL, about 223 μL, about 224 μL, about 225 μL, about 226 μL, about 227 μL, about 228 μL, about 229 μL, about 230 μL, about 231 μL, about 232 μL, about 233 μL, about 234 μL, about 235 μL, about 236 μL, about 237 μL, about 238 μL, about 239 μL, about 240 μL, about 241 μL, about 242 μL, about 243 μL, about 244 μL, about 245 μL, about 246 μL, about 247 μL, about 248 μL, about 249 μL, about 250 μL, about 251 μL, about 252 μL, about 253 μL, about 254 μL, about 255 μL, about 256 μL, about 257 μL, about 258 μL, about 259 μL, about 260 μL, about 261 μL, about 262 μL, about 263 μL, about 264 μL, about 265 μL, about 266 μL, about 267 μL, about 268 μL, about 269 μL, about 270 μL, about 271 μL, about 272 μL, about 273 μL, about 274 μL, about 275 μL, about 276 μL, about 277 μL, about 278 μL, about 279 μL, about 280 μL, about 281 μL, about 282 μL, about 283 μL, about 284 μL, about 285 μL, about 286 μL, about 287 μL, about 288 μL, about 289 μL, about 290 μL, about 291 μL, about 292 μL, about 293 μL, about 294 μL, about 295 μL, about 296 μL, about 297 μL, about 298 μL, about 299 μL, 300 μL, about 305 μL, about 310 μL, about 315 μL, about 320 μL, about 325 μL, about 330 μL, about 335 μL, about 340 μL, about 345 μL, about 350 μL, about 355 μL, about 360 μL, about 365 μL, about 370 μL, about 375 μL, about 380 μL, about 385 μL, about 390 μL, about 395 μL, about 400 μL, about 405 μL, about 410 μL, about 415 μL, about 420 μL, about 425 μL, about 430 μL, about 435 μL, about 440 μL, about 445 μL, about 450 μL, about 455 μL, about 460 μL, about 465 μL, about 470 μL, about 475 μL, about 480 μL, about 485 μL, about 490 μL, about 495 μL, about 500 μL, about 505 μL, about 510 μL, about 515 μL, about 520 μL, about 525 μL, about 530 μL, about 535 μL, about 540 μL, about 545 μL, about 550 μL, about 555 μL, about 560 μL, about 565 μL, about 570 μL, about 575 μL, about 580 μL, about 585 μL, about 590 μL, about 595 μL, about 600 μL, about 605 μL, about 610 μL, about 615 μL, about 620 μL, about 625 μL, about 630 μL, about 635 μL, about 640 μL, about 645 μL, about 650 μL, about 655 μL, about 660 μL, about 665 μL, about 670 μL, about 675 μL, about 680 μL, about 685 μL, about 690 μL, about 695 μL, about 700 μL, about 705 μL, about 710 μL, about 715 μL, about 720 μL, about 725 μL, about 730 μL, about 735 μL, about 740 μL, about 745 μL, about 750 μL, about 755 μL, about 760 μL, about 765 μL, about 770 μL, about 775 μL, about 780 μL, about 785 μL, about 790 μL, about 795 μL, about 800 μL, about 805 μL, about 810 μL, about 815 μL, about 820 μL, about 825 μL, about 830 μL, about 835 μL, about 840 μL, about 845 μL, about 850 μL, about 855 μL, about 860 μL, about 865 μL, about 870 μL, about 875 μL, about 880 μL, about 885 μL, about 890 μL, about 895 μL, about 900 μL, about 905 μL, about 910 μL, about 915 μL, about 920 μL, about 925 μL, about 930 μL, about 935 μL, about 940 μL, about 945 μL, about 950 μL, about 955 μL, about 960 μL, about 965 μL, about 970 μL, about 975 μL, about 980 μL, about 985 μL, about 990 μL, about 995 μL, or about 1000 μL.

In some embodiments, the first liquid stream and the second liquid stream are contacted (or mixed) at about room temperature (e.g., at about 20-22° C.). In some embodiments, the first liquid stream and the liquid second stream are contacted (or mixed) at a temperature of about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46, about 47° C., about 48° C., about 49° C., about 50° C., about 51° C., about 52° C., about 53° C., about 54° C., about 55° C., about 56° C., about 57° C., about 58° C., about 59° C., or about 60° C. In some embodiments, the first liquid stream and the second liquid stream are contacted (or mixed) at 25° C.+/−5° C. In some embodiments, the first liquid stream and the second liquid stream are contacted (or mixed) at 20° C.+/−10° C.

In some embodiments, the isolating comprises conveying the polyplex (e.g., as it forms after mixing the polymer and nucleic acid component) in a liquid channel for a residence time sufficient to stabilize the polyplex.

In some embodiments, the residence time is from about 1 second (sec) to about 20 minutes (min), including about 1 sec, about 2 sec, about 3 sec, about 4 sec, about 5 sec, about 6 sec, about 7 sec, about 8 sec, about 9 sec about 10 sec, about 15 sec, about 20 sec, about 30 sec, about 40 sec, about 50 sec, about 1 min, about 1.5 min, about 2 min, about 2.5 min, about 3 min, about 3.5 min, about 4 min, about 4.5 min, about 5 min, about 5.5 min, about 6 min, about 6.5 min, about 7 min, about 7.5 min, about 8 min, about 8.5 min, about 9 min, about 9.5 min, about 10 min, about 10.5 min, about 11 min, about 11.5 min, about 12 min, about 12.5 min, about 13 min, about 13.5 min, about 14 min, about 14.5 min, about 15.0 min, about 15.5 min, about 16 min, about 16.5 min, about 17 min, about 17.5 min, about 18 min, about 18.5 min, about 19 min, about 19.5 min, and about 20 min including all ranges there between.

In some embodiments, the residence time is about 1 sec, about 2 sec, about 3 sec, about 4 sec, about 5 sec, about 6 sec, about 7 sec, about 8 sec, about 9 sec about 10 sec, about 15 sec, about 20 sec, about 30 sec, about 40 sec, about 50 sec, about 1 min, about 1.5 min, about 2 min, about 2.5 min, about 3 min, about 3.5 min, about 4 min, about 4.5 min, about 5 min, about 5.5 min, about 6 min, about 6.5 min, about 7 min, about 7.5 min, about 8 min, about 8.5 min, about 9 min, about 9.5 min, about 10 min, about 10.5 min, about 11 min, about 11.5 min, about 12 min, about 12.5 min, about 13 min, about 13.5 min, about 14 min, about 14.5 min, about 15.0 min, about 15.5 min, about 16 min, about 16.5 min, about 17 min, about 17.5 min, about 18 min, about 18.5 min, about 19 min, about 19.5 min, or about 20 min.

In some embodiments, the residence time is no less than 1 second and no more than 10 minutes. In some embodiments, the residence time is about 10 minutes.

Different polyplex products (i.e., different combinations of polymers and nucleic acid components) may require different settling or formation times. Settling and formation times may be varied by lengthening or shortening the residence time in the liquid channel

In some embodiments, the methods disclosed herein prevent the newly formed polyplexes from clumping, agglomerating, clotting, grouping, or sticking together. For example, by contacting the first and second liquid streams in a pulsed manner, the resulting polyplexes can be spatially separated from each other during formation and/or prior to isolation.

In some embodiments, the liquid channel is slightly negatively charged. In some embodiments, the liquid channel comprises an aqueous phase surrounded by a carrier fluid. In some embodiments, the density of the carrier fluid is a value in the range of about 1,300 kg/m³ to about 2,000 kg/m³ and the density of the aqueous phase is a value in the range of about 900 kg/m³ to about 1200 kg/m³. In some embodiments the density of the carrier fluid is about 1,300 kg/m³, about 1,400 kg/m³, about 1,500 kg/m³, about 1,600 kg/m³, about 1,700 kg/m³, about 1,800 kg/m³, about 1,900 kg/m³, or about 2,000 kg/m³, including all ranges between any of the values herein. In some embodiments, the density of the aqueous phase is about 900 kg/m³, about 1,000 kg/m³, about 1,100 kg/m³, or about 1,200 kg/m³ including all ranges between any of the values herein. In some embodiments, the aqueous phase comprises sodium acetate buffer.

In some embodiments, the carrier fluid is an oil (as described herein). In some embodiments, the oil channel is about 5-100 mm wide. Without wishing to be bound by theory, the slightly negative charge of the oil/liquid channel electrostatically attracts the positively charged polyplexes, conveying the polyplexes in an orderly fashion through the liquid channel and enhancing the stability of the system.

In some embodiments, the oil is selected from the group consisting of: (a) Fluorinert FC-40 (fluorocarbonated oil), (b) silicon oil, (c) mineral oil, (d) perfluorinated amine oil, (e) phenylmethylpolysiloxane or (f) phenylmethylpolysiloxane-based oil and an additive. In some embodiments, the additive has a hydrophilic-lipophilic balance number in the range of 2 to 8. In some embodiments, the additive is a polysorbate additive.

In some embodiments, the polysorbate additive is SPAN 80 (sorbitan oleate), SPAN 65 (sorbitan tristearate) or Tween 20 (polyethylene glycol sorbitan monolaurate).

In some embodiments, the concentration of the additive in the aqueous phase is from about 0.001% and about 10% (wt/wt %).

In some embodiments, the additives are added to a final concentration of between 10 mg/mL to 50 mg/mL in the aqueous phase. In one aspect, the polyplexes are spatially separated or segregated in the liquid channel. In some embodiments, the space between the segregated polyplexes is controlled by the velocity of liquid in the channel That is, because the individual polyplexes are formed by contacting a portion of DNA solution and a portion of polymer solution at a discrete rate before being introduced into the liquid channel, the greater the velocity of the liquid in the liquid channel, the smaller the spatial separation between polyplexes.

In some embodiments, the velocity of the liquid moving in the channel is from about 0.1 m/s to about 1 m/s, including about 0.1 m/s, about 0.11 m/s, about 0.12 m/s, about 0.13 m/s, about 0.14 m/s, about 0.15 m/s, about 0.16 m/s, about 0.17 m/s, about 0.18 m/s, about 0.19 m/s, about 0.2 m/s, about 0.21 m/s, about 0.22 m/s, about 0.23 m/s, about 0.24 m/s, about 0.25 m/s, about 0.26 m/s, about 0.27 m/s, about 0.28 m/s, about 0.29 m/s, about 0.30 m/s, about 0.31 m/s, about 0.32 m/s, about 0.33 m/s, about 0.34 m/s, about 0.35 m/s, about 0.36 m/s, about 0.37 m/s, about 0.38 m/s, about 0.39 m/s, about 0.4 m/s, about 0.41 m/s, about 0.42 m/s, about 0.43 m/s, about 0.44 m/s, about 0.45 m/s, about 0.46 m/s, about 0.47 m/s, about 0.48 m/s, about 0.49 m/s, about 0.5 m/s, about 0.51 m/s, about 0.52 m/s, about 0.53 m/s, about 0.54 m/s, about 0.55 m/s, about 0.56 m/s, about 0.57 m/s, about 0.57 m/s, about 0.59 m/s, about 0.6 m/s, about 0.61 m/s, about 0.62 m/s, about 0.63 m/s, about 0.64 m/s, about 0.65 m/s, about 0.66 m/s, about 0.67 m/s, about 0.68 m/s, about 0.69 m/s, about 0.7 m/s, about 0.71 m/s, about 0.72 m/s, about 0.73 m/s, about 0.74 m/s, about 0.75 m/s, about 0.76 m/s, about 0.77 m/s, about 0.78 m/s, about 0.79 m/s, about 0.8 m/s, about 0.81 m/s, about 0.82 m/s, about 0.83 m/s, about 0.84 m/s, about 0.85 m/s, about 0.86 m/s, about 0.87 m/s, about 0.88 m/s, about 0.9 m/s, about 0.91 m/s, about 0.92 m/s, about 0.93 m/s, about 0.94 m/s, about 0.95 m/s, about 0.96 m/s, about 0.97 m/s, about 0.98 m/s, and about 1.0 m/s, including all ranges there between.

In some embodiments, the velocity of the liquid moving in the channel is about 0.10 m/s, about 0.11 m/s, about 0.12 m/s, about 0.13 m/s, about 0.14 m/s, about 0.15 m/s, about 0.16 m/s, about 0.17 m/s, about 0.18 m/s, about 0.19 m/s, about 0.20 m/s, about 0.21 m/s, about 0.22 m/s, about 0.23 m/s, about 0.24 m/s, about 0.25 m/s, about 0.26 m/s, about 0.27 m/s, about 0.28 m/s, about 0.29 m/s, about 0.30 m/s, about 0.31 m/s, about 0.32 m/s, about 0.33 m/s, about 0.34 m/s, about 0.35 m/s, about 0.36 m/s, about 0.37 m/s, about 0.38 m/s, about 0.39 m/s, about 0.40 m/s, about 0.41 m/s, about 0.42 m/s, about 0.43 m/s, about 0.44 m/s, about 0.45 m/s, about 0.46 m/s, about 0.47 m/s, about 0.48 m/s, about 0.49 m/s, about 0.50 m/s, about 0.51 m/s, about 0.52 m/s, about 0.53 m/s, about 0.54 m/s, about 0.55 m/s, about 0.56 m/s, about 0.57 m/s, about 0.58 m/s, about 0.59 m/s, about 0.60 m/s, about 0.61 m/s, about 0.62 m/s, about 0.63 m/s, about 0.64 m/s, about 0.65 m/s, about 0.66 m/s, about 0.67 m/s, about 0.68 m/s, about 0.69 m/s, about 0.70 m/s, about 0.71 m/s, about 0.71 m/s, about 0.72 m/s, about 0.73 m/s, about 0.74 m/s, about 0.75 m/s, about 0.76 m/s, about 0.77 m/s, about 0.78 m/s, about 0.78 m/s, about 0.79 m/s, above 0.80 m/s, about 0.81 m/s, about 0.82 m/s, about 0.83 m/s, about 0.84 m/s, about 0.85 m/s, about 0.86 m/s, about 0.87 m/s, about 0.88 m/s, about 0.89 m/s, about 0.90 m/s, about 0.91 m/s, about 0.92 m/s, about 0.93 m/s, about 0.94 m/s, about 0.95 m/s, about 0.96 m/s, about 0.97 m/s, about 0.98 m/s, about 0.99 m/s, or about 1.0 m/s.

In some embodiments, the fluid stream in which the polyplexes are formed moves under laminar flow conditions. In some embodiments, the fluid stream moves under partial laminar flow conditions.

In some embodiments, the method further comprises assessing and harvesting one or more polyplexes.

In some embodiments, assessing the polyplexes occurs (e.g., by an in line QC test) while the polyplexes are moving in the liquid channel to determine if the polyplexes conform to specification. For example, in some embodiments, a region of interest in the liquid channel and the polyplexes therein is monitored by high-speed cameras, which measure the optical characteristics (e.g., refractive index) of the polyplexes. Polyplexes that fail to meet specification are separated and directed to waste. In some embodiments, the polyplexes that fail to meet specification are separated by applying a pneumatic pressure to the region of interest. The polyplexes that meet specification are harvested.

In some embodiments, assessing comprises measuring the refractive index of polyplexes in the liquid channel; and optionally removing polyplexes with a refractive index that does not conform to specification. The refractive index of the polyplex is correlated with desirable attributes of a properly formed polyplex (such as, a desired polyplex particle size).

In some embodiments, when the polyplex particles are fully formed, the polyplex particles and the oil/liquid channel enter an area where the negative charge is removed from the oil/liquid allowing the particles to be easily separated from the oil/liquid and harvested.

In some embodiments, harvesting the stabilized polyplexes comprises:

a) collecting the polyplexes in a reservoir; or
b) collecting an aliquot of a defined amount of liquid polyplexes, and freezing the aliquot for therapeutic use; or
c) collecting the polyplexes in a receptacle for the freezing or freeze drying of the polyplexes in preparation for the final therapeutic product.

In some embodiments, the method further comprises filtering, washing, freezing and/or lyophilizing the stabilized polyplexes.

In some embodiments, the method further comprises first filtering and washing the polyplexes.

In some embodiments, the method comprises freezing and/or lyophilizing the polyplexes.

In some embodiments, the lyophilized polyplexes can be packaged and reliably stored.

In some embodiments, the polymer is positively charged and the nucleic acid component is negatively charged.

In some embodiments, the polymer is first dissolved in an organic solvent at a concentration of no more than 300 μg/μL, and then suspended in sodium acetate solution. In some embodiments, the sodium acetate solution is 25 mM and pH 5.2

In some embodiments, the concentration of the polymer in an organic solvent is from about 1 μg/μL, to about 300 μg/μL, including about 1 μg/μL, about 5 μg/μL, about 10 μg/μL, about 15 μg/μL, about 20 μg/μL, about 25 μg/μL, about 30 μg/μL, about 35 μg/μL, about 40 μg/μL, about 45 μg/μL, about 50 μg/μL, about 55 μg/μL, about 60 μg/μL, about 65 μg/μL, about 70 μg/μL, about 75 μg/μL, about 80 μg/μL, about 85 μg/μL, about 90 μg/μL, about 95 μg/μL, about 100 μg/μL, about 105 μg/μL, about 110 μg/4, about 115 μg/μL, about 120 μg/μL, about 125 μg/μL, about 130 μg/μL, about 135 μg/μL, about 140 μg/μL, about 145 μg/μL, about 150 μg/μL, about 155 μg/μL, about 160 μg/μL, about 165 μg/μL, about 170 μg/μL, about 175 μg/μL, about 180 μg/μL, about 185 μg/μL, about 190 μg/μL, about 195 μg/μL, about 200 μg/μL, about 205 μg/μL, about 210 μg/μL, about 215 μg/μL, about 220 μg/μL, about 225 μg/μL, about 230 μg/μL, about 235 μg/μL, about 240 μg/μL, about 245 μg/μL, about 250 μg/μL, about 255 μg/μL, about 260 μg/4, about 265 μg/μL, about 270 μg/μL, about 275 μg/μL, about 280 μg/μL, about 285 μg/μL, about 290 μg/μL, about 295 μg/μL, and about 300 μg/μL, including all ranges there between.

In some embodiments, the concentration of the polymer in an organic solvent is about 1 μg/μL, about 2 μg/μL, about 3 μg/μL, about 4 μg/μL, about 5 μg/μL, about 6 μg/μL, about 7 μg/μL, about 8 μg/μL, about 9 μg/μL, about 10 μg/μL, about 11 μg/μL, about 12 μg/μL, about 13 μg/μL, about 14 μg/μL, about 15 μg/μL, about 16 μg/μL, about 17 μg/μL, about 18 μg/μL, about 19 μg/μL, about 20 μg/μL, about 21 μg/μL, about 22 μg/μL, about 23 μg/μL, about 24 μg/μL, about 25 μg/μL, about 26 μg/μL, about 27 μg/μL, about 28 μg/μL, about 29 μg/μL, about 30 μg/μL, about 31 μg/μL, about 32 μg/μL, about 33 μg/μL, about 34 μg/μL, about 35 μg/μL, about 36 μg/μL, about 37 μg/μL, about 38 μg/μL, about 39 μg/μL, about 40 μg/μL, about 41 μg/μL, about 42 μg/μL, about 43 μg/μL, about 44 μg/μL, about 45 μg/μL, about 46 μg/μL, about 47 μg/μL, about 48 μg/μL, about 49 μg/μL, about 50 μg/μL, about 51 μg/μL, about 52 μg/μL, about 53 μg/μL, about 54 μg/μL, about 55 μg/μL, about 56 μg/μL, about 57 μg/μL, about 58 μg/μL, about 59 μg/μL, about 60 μg/μL, about 61 μg/μL, about 62 μg/μL, about 63 μg/μL, about 64 μg/μL, about 65 μg/μL, about 67 μg/μL, about 68 μg/μL about 69 μg/μL about 70 μg/μL about 71 μg/μL, about 72 μg/μL about 73 μg/μL about 74 μg/μL about 75 μg/μL about 76 μg/μL, about 77 μg/μL about 78 μg/μL about 79 μg/μL about 80 μg/μL about 81 μg/μL about 82 μg/μL, about 83 μg/μL about 84 μg/μL about 85 μg/μL about 86 μg/μL about 87 μg/μL, about 88 μg/μL about 89 μg/μL about 90 μg/μL about 91 μg/μL about 92 μg/μL about 93 μg/μL, about 94 μg/μL about 95 μg/μL about 96 μg/μL about 97 μg/μL about 98 μg/μL, about 99 μg/μL about 100 μg/μL about 101 μg/μL about 102 μg/μL, about 103 μg/μL, about 104 μg/μL about 105 μg/μL, about 106 μg/μL about 107 μg/μL, about 108 μg/μL, about 109 μg/μL about 110 μg/μL, about 111 μg/μL about 112 μg/μL, about 113 μg/μL, about 114 μg/μL about 115 μg/μL, about 116 μg/μL about 117 μg/μL, about 118 μg/μL, about 119 μg/μL about 120 μg/μL, about 121 μg/μL about 122 μg/μL, about 123 μg/μL, about 124 μg/μL about 125 μg/μL, about 126 μg/μL about 127 μg/μL, about 128 μg/μL, about 129 μg/μL about 130 μg/μL, about 131 μg/μL about 132 μg/μL, about 133 μg/μL, about 134 μg/μL about 135 μg/μL, about 136 μg/μL about 137 μg/μL, about 138 μg/μL, about 139 μg/μL about 140 μg/μL, about 141 μg/μL about 142 μg/μL, about 143 μg/μL, about 144 μg/μL about 145 μg/μL, about 146 μg/μL, about 147 μg/μL about 148 μg/μL about 149 μg/μL, about 150 μg/μL, about 151 μg/μL about 152 μg/μL about 153 μg/μL about 154 μg/μL about 155 μg/μL, about 156 μg/μL about 157 μg/μL about 158 μg/μL about 159 μg/μL about 160 μg/μL, about 161 μg/μL about 162 μg/μL about 163 μg/μL about 164 μg/μL about 165 μg/μL, about 166 μg/μL about 167 μg/μL about 168 μg/μL about 169 μg/μL about 170 μg/μL, about 171 μg/μL about 172 μg/μL about 173 μg/μL about 174 μg/μL about 175 μg/μL, about 176 μg/μL about 177 μg/μL about 178 μg/μL about 179 μg/μL about 180 μg/μL, about 181 μg/μL about 182 μg/μL about 183 μg/μL about 184 μg/μL about 185 μg/μL, about 186 μg/μL about 187 μg/μL about 188 μg/μL about 189 μg/μL about 190 μg/μL, about 191 μg/μL about 192 μg/μL about 193 μg/μL about 194 μg/μL about 195 μg/μL, about 196 μg/μL about 197 μg/μL about 198 μg/μL about 199 μg/μL about 200 μg/μL, about 201 μg/μL about 202 μg/μL about 203 μg/μL about 204 μg/μL about 205 μg/μL, about 206 μg/μL about 207 μg/μL about 208 μg/μL about 209 μg/μL about 210 μg/μL, about 211 μg/μL about 212 μg/μL about 213 μg/μL about 214 μg/μL about 215 μg/μL, about 216 μg/μL about 217 μg/μL about 218 μg/μL about 219 μg/μL about 220 μg/μL, about 221 μg/μL about 222 μg/μL about 223 μg/μL about 224 μg/μL about 225 μg/μL, about 226 μg/μL about 227 μg/μL about 228 μg/μL about 229 μg/μL about 230 μg/μL, about 231 μg/μL about 232 μg/μL about 233 μg/μL about 234 μg/μL about 235 μg/μL, about 236 μg/μL about 237 μg/μL about 238 μg/μL about 239 μg/μL about 240 μg/μL, about 241 μg/μL, about 242 μg/μL, about 243 μg/μL, about 244 μg/μL, about 245 μg/μL, about 246 μg/μL, about 247 μg/μL, about 248 μg/μL, about 249 μg/μL, about 250 μg/μL, about 251 μg/μL, about 252 μg/μL, about 253 μg/μL, about 254 μg/μL, about 255 μg/μL, about 256 μg/μL, about 257 μg/μL, about 258 μg/μL, about 259 μg/μL, about 260 μg/μL, about 261 μg/μL, about 262 μg/μL, about 263 μg/μL, about 264 μg/μL, about 265 μg/μL, about 266 μg/μL, about 267 μg/μL, about 268 μg/μL, about 269 μg/μL, about 270 μg/μL, about 271 μg/μL, about 272 μg/μL, about 273 μg/μL, about 274 μg/μL, about 275 μg/μL, about 276 μg/μL, about 277 μg/μL, about 278 μg/μL, about 279 μg/μL, about 280 μg/μL, about 281 μg/μL, about 282 μg/μL, about 283 μg/μL, about 284 μg/μL, about 285 μg/μL, about 286 μg/μL, about 287 μg/μL, about 288 μg/μL, about 289 μg/μL, about 290 μg/μL, about 291 μg/μL, about 292 μg/μL, about 293 μg/μL, about 294 μg/μL, about 295 μg/μL, about 296 μg/μL, about 297 μg/μL, about 298 μg/μL, about 299 μg/μL, or about 300 μg/μL.

In some embodiments, the organic solvent used to initially dissolve the polymer is a water miscible organic solvent. In some embodiments, the organic solvent is dimethyl sulfoxide (DMSO), dimethylformamide (DMF), acetonitrile, tetrahydrofuran, methanol, ethanol, or isopropanol. In some embodiments, the organic solvent is DMSO, N,N-dimethylformamide (DMF), N-methylpyrrolidone (NMP) and the like, ketones such as acetone, methyl ethyl ketone, methyl isobutyl ketone and the like; or ethers such tetrahydrofuran (THF). In some embodiments, the organic solvent is DMSO.

In some embodiments, the nucleic acid component (such as DNA) is first dissolved in a solvent at a concentration of no more than 4 mg/mL and then suspended in sodium acetate solution. In some embodiments, the sodium acetate solution is 25 mM and pH 5.2.

In some embodiments, the nucleic acid component (such as DNA) is dissolved to provide a solution having a concentration of no more than about 4 mg/mL, including no more than about 3.5 mg/mL, no more than about 3.0 mg/mL, no more than about 2.5 mg/mL, no more than about 2.0 mg/mL, no more than about 1.5 mg/mL, no more than about 1.0 mg/mL, no more than about 0.5 mg/mL, no more than about 0.1 mg/mL, no more than about 0.05 mg/mL, and no more than about 0.01 mg/mL; including all concentrations in between.

In some embodiments, the nucleic acid component (such as DNA) is dissolved to provide a solution having a concentration of about 0.01 mg/mL to 4 mg/mL, including about 0.01 mg/mL, about 0.02 mg/mL, about 0.06 mg/mL, about 0.07 mg/mL, about 0.08 mg/mL, about 0.09 mg/mL, about 0.1 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, about 0.20 mg/mL, about 0.25 mg/mL, about 0.30 mg/mL, about 0.35 mg/mL, about 0.40 mg/mL, about 0.45 mg/mL, about 0.50 mg/mL, about 0.55 mg/mL, about 0.60 mg/mL, about 0.65 mg/mL, about 0.70 mg/mL, about 0.75 mg/mL, about 0.80 mg/mL, about 0.85 mg/mL, about 0.90 mg/mL, about 0.95 mg/mL, about 1.0 mg/mL, about 1.05 mg/mL, about 1.10 mg/mL, about 1.15 mg/mL, about 1.20 mg/mL, about 1.25 mg/mL, about 1.30 mg/mL, about 1.35 mg/mL, about 1.40 mg/mL, about 1.45 mg/mL, about 1.50 mg/mL, about 1.55 mg/mL, about 1.60 mg/mL, about 1.65 mg/mL, about 1.70 mg/mL, about 1.75 mg/mL, about 1.80 mg/mL, about 1.85 mg/mL, about 1.90 mg/mL, about 1.95 mg/mL, about 2.0 mg/mL, about 2.10 mg/mL, about 2.15 mg/mL, about 2.20 mg/mL, about 2.25 mg/mL, about 2.30 mg/mL, about 2.35 mg/mL, about 2.40 mg/mL, about 2.45 mg/mL, about 2.50 mg/mL, about 2.55 mg/mL, about 2.60 mg/mL, about 2.65 mg/mL, about 2.70 mg/mL, about 2.75 mg/mL, about 2.80 mg/mL, about 2.85 mg/mL, about 2.90 mg/mL, about 2.95 mg/mL, about 3.0 mg/mL, about 3.10 mg/mL, about 3.15 mg/mL, about 3.20 mg/mL, about 3.25 mg/mL, about 3.30 mg/mL, about 3.35 mg/mL, about 3.40 mg/mL, about 3.45 mg/mL, about 3.50 mg/mL, about 3.55 mg/mL, about 3.60 mg/mL, about 3.65 mg/mL, about 3.70 mg/mL, about 3.75 mg/mL, about 3.80 mg/mL, about 3.85 mg/mL, about 3.90 mg/mL, about 3.95 mg/mL, and about 4.0 mg/mL including all values there between. In some embodiments, the polymer and the nucleic acid component (such as DNA) is suspended in a liquid containing a sugar. For example, trehalose, sucrose, glucose and mannitol. In some embodiments, the sugar is sucrose.

In some embodiments, the method of the present disclosure employs any polymer capable of forming a polyplex, including polyethyleneimines, linear poly(amino esters) (LPAEs) [e.g., those described in Mol Ther. 2005, 11(3), 426-434; and Methods Mol Biol. 2009; 480, 53-63, the contents of which are each hereby incorporated by reference in their entirety], chitosan, poly(lactic-co-glycolic acid) polymers, polylactic acid polymers, and highly branched poly(amino esters), such as highly branched poly(b-amino esters) [e.g., those described in Biomacromolecules 2016, 17, 3640-3647; Biomacromolecules 2015, 16, 2609-2617; ACS Appl. Mater. Interfaces 2016, 8, 50, 34218-34226; J Control Release, 2016, 244 (Pt B), 336-346; Drug Discov. Today. 2013 November; 18 (21-22), 1090-1098; Sci. Adv. 2016; 2: e1600102; WO 2014/053654; US 2015/0266986; and WO 2016/020474 the contents of which are each hereby incorporated by reference in their entirety]. In some embodiments, the polymer is a highly branched poly(amino ester) (HPAE) for example as described below.

In one aspect, the method described herein, provide a polyplex comprising a nucleic acid component and a polymer comprising:

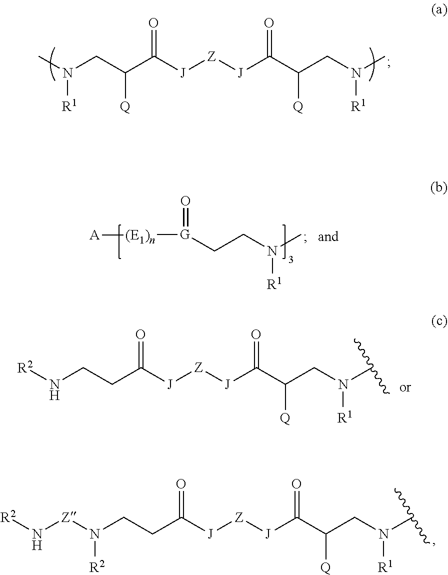

each J is —O—

Z″ is a linking moiety;

Z, is a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms; or Z is

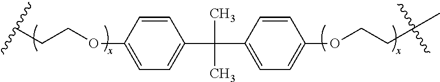

wherein xis 1-1000;

wherein Z is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a haloC₁-C₆ alkyl, a carboxyl group, a cyano group, a nitro group, a nitroso group, —OC(O)NR′R′, —N(R′)C(O)NR′R′, —N(V)C(O)O—C₁-C₆alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—;

each Q is H;

each E₁ is heteroalkylene;

R₁ and R₂ are each independently C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; wherein R₁ and R₂ are each independently unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1

In some embodiments, Z, is a linear or branched carbon chain of 1 to 30 carbon atoms, or a linear or branched heteroatom-containing carbon chains of 1 to 30 atoms; wherein Z is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆thioether, a C₁-C₆ sulfone, a C₁-C₆ sulfoxide, a C₁-C₆primary amide, a C₁-C₆ secondary amide, a haloC₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl; or Z is

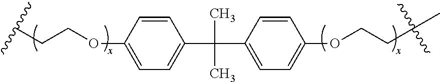

wherein x is 1-1000.

In some embodiments, Z″ is a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms; wherein Z is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a haloC₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl

In some embodiments, Z″ is a linear carbon chain of 1 to 30 carbon atoms. For example, Z″ may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like.

In some embodiments, Z″ is a linear or branched carbon chain of 1 to 30 carbon atoms or a linear or branched heteroatom-containing carbon chains of 1 to 30 atoms.

In some embodiments, Z is a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms; wherein Z is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl.

In some embodiments, Z is a linear or branched carbon chain of 1 to 30 carbon atoms or a linear or branched heteroatom-containing carbon chains of 1 to 30 atoms. In some embodiments, Z is a linear carbon chain of 1 to 30 carbon atoms. For example, Z may be an alkylene group including but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z is a branched carbon chain of 1 to 30 carbon atoms. In some embodiments, Z is a linear or branched heteroatom-containing carbon chain of 1 to 30 atoms. For example, Z may be a linear or branched carbon chain with one or more of the carbon atoms substituted with a heteroatom, including but not limited to O, N, S, or P. In some embodiments, Z is a linear or branched carbon chain of 1 to 10 carbon atoms. In some embodiments, Z is

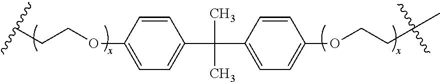

wherein x is 1-1000. In some embodiments, Z is

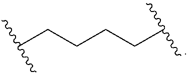 [SMILES: *CCCC*]

In some embodiments, R₁ and R₂ are independently C₁-C₂₀ alkyl. For example, R₁ and R₂ may be C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl groups such as such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-icosyl. In some embodiments, R₁ and R₂ are independently unsubstituted. In some embodiments, R₁ and R₂ are independently substituted. In some embodiments R₁ and R₂ are independently selected from the group consisting of

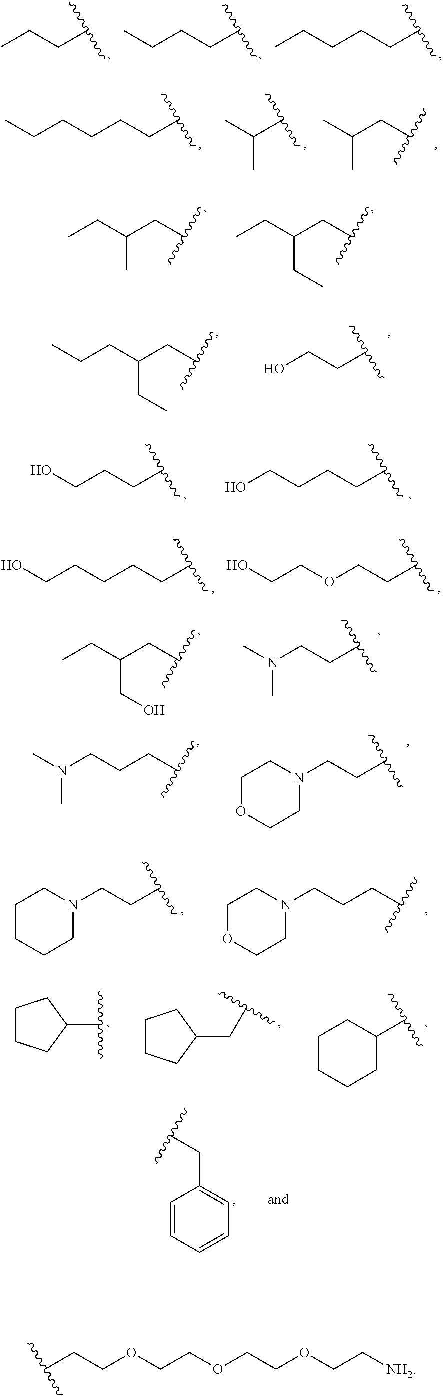

In some embodiments, R₁ is

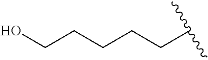 [SMILES: *CCCCCO]

### and R₂ is

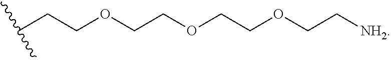 [SMILES: *CCOCCOCCOCCN]

In some embodiments, R₁ is

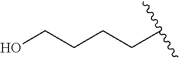 [SMILES: *CCCCO]

### and R₂ is

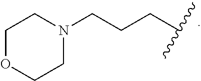 [SMILES: *CCCN1CCOCC1]

In some embodiments, the polymer comprises:

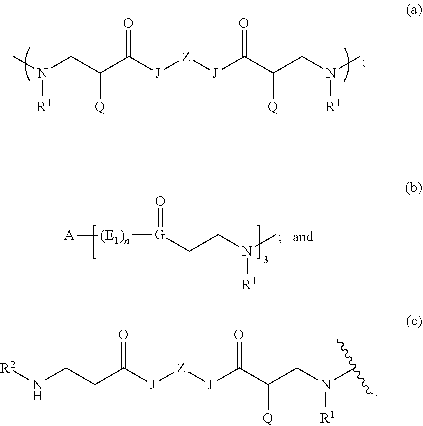

In some embodiments, the polymer comprises:

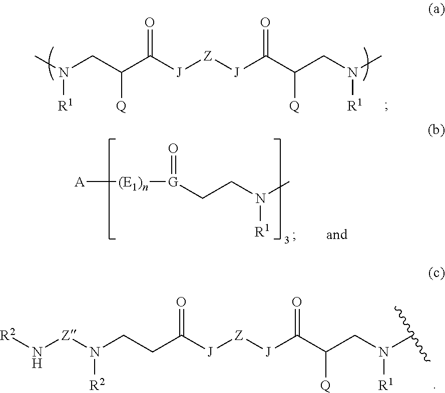

In some embodiments, the polymer comprises:

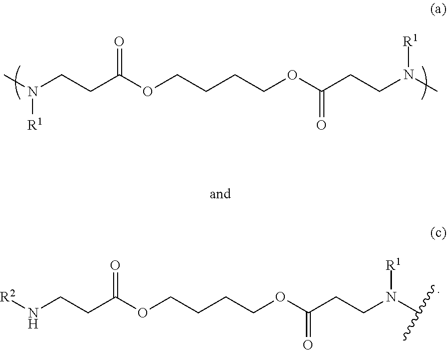

In some embodiments, the polymer comprises:

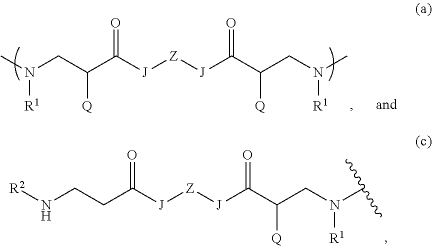

wherein

### J is O and Z is

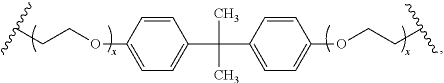

wherein x is 1-1000.

In some embodiments, the polymer comprises:

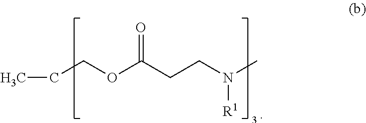

In some embodiments, the polymer comprises:

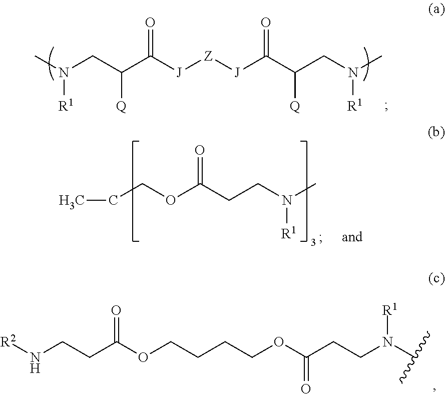

wherein

### R₁ is

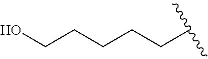 [SMILES: *CCCCCO]

and
R₂ is selected from

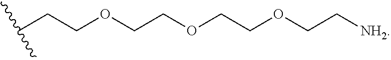 [SMILES: *CCOCCOCCOCCN]

In some embodiments, the polyplex comprises a nucleic acid component and a polymer comprising:

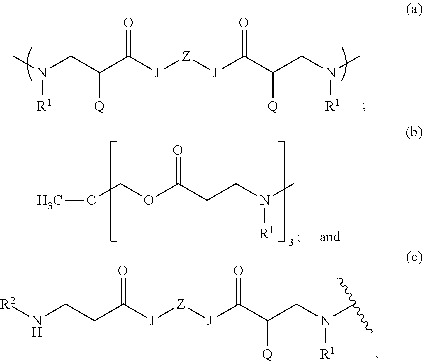

wherein

J is O and Z is

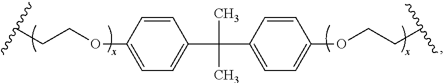

wherein x is 1-1000;

R₁ is

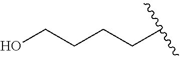 [SMILES: *CCCCO]

and

R₂ is

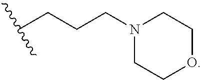 [SMILES: *CCCN1CCOCC1]

In some embodiments, the polymers of the present disclosure have an alpha parameter defined from the Mark-Houwink equation of less than about 0.5. For example, the polymers of the present disclosure have an alpha parameter defined from the Mark-Houwink equation ranging from about 0.01 to about 0.49. For example, the polymers of the present disclosure have an alpha parameter defined from the Mark-Houwink equation ranging from about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about to about 0.49, including all ranges there between. In some embodiments, the polymers of the present disclosure have an alpha parameter defined from the Mark-Houwink equation from about 0.2 to about 0.5.

In some embodiments, the polymers of the present disclosure have an alpha parameter defined from the Mark-Houwink equation of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, or about 0.49.

In some embodiments, the polymer has an alpha parameter defined from the Mark-Houwink equation of less than about 0.5. In some embodiments, the polymer has an alpha parameter defined from the Mark-Houwink equation from about 0.2 to about 0.5.

The term “polydispersity index” (PDI) refers to a measure of the distribution of molecular mass in a given polymer sample. The polydispersity index is calculated by dividing the weight average molecular weight (Mw) by the number average molecular weight (Mn). As used herein, the term “weight average molecular weight” generally refers to a molecular weight measurement that depends on the contributions of polymer molecules according to their sizes. As used herein, the term “number average molecular weight” generally refers to a molecular weight measurement that is calculated by dividing the total weight of all the polymer molecules in a sample with the total number of polymer molecules in the sample. These terms are well-known by those of ordinary skill in the art.

In some embodiments, the polymers of the present disclosure have a PDI from about 1.01 to about 8.0. For example, the PDI may range from about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, to about 8.0, including all ranges there between.

In some embodiments, the polymers of the present disclosure have a PDI of about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the polymers of the present disclosure have a PDI of about 2.5.

In some embodiments, the polymers of the present disclosure have a PDI from about 1.01 to about 8.0. In some embodiments, the polymers of the present disclosure have a PDI of about 2.5.

In some embodiments, the polymers of the present disclosure have a Mw of at least 3 kDa. In some embodiments, the polymers of the present disclosure have a Mw of about 3 kDa to about 200 kDa. Accordingly, the polymers of the present disclosure have a Mw ranging from about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, about 80 kDa, about 81 kDa, about 82 kDa, about 83 kDa, about 84 kDa, about 85 kDa, about 86 kDa, about 87 kDa, about 88 kDa, about 89 kDa, about 90 kDa, about 91 kDa, about 92 kDa, about 93 kDa, about 94 kDa, about 95 kDa, about 96 kDa, about 97 kDa, about 98 kDa, about 99 kDa, about 100 kDa about, 101 kDa, about 102 kDa, about 103 kDa, about 104 kDa, about 105 kDa, about 106 kDa, about 107 kDa, about 108 kDa, about 109 kDa, about 110 kDa, about 111 kDa, about 112 kDa, about 113 kDa, about 114 kDa, about 115 kDa, about 116 kDa, about 117 kDa, about 118 kDa, about 119 kDa, about 120 kDa, about 121 kDa, about 122 kDa, about 123 kDa, about 124 kDa, about 125 kDa, about 126 kDa, about 127 kDa, about 128 kDa, about 129 kDa, about 130 kDa, about 131 kDa, about 132 kDa, about 133 kDa, about 134 kDa, about 135 kDa, about 136 kDa, about 137 kDa, about 138 kDa, about 139 kDa, about 140 kDa, about 141 kDa, about 142 kDa, about 143 kDa, about 144 kDa, about 145 kDa, about 146 kDa, about 147 kDa, about 148 kDa, about 149 kDa, about 150 kDa, about 151 kDa, about 152 kDa, about 153 kDa, about 154 kDa, about 155 kDa, about 156 kDa, about 157 kDa, about 158 kDa, about 159 kDa, about 160 kDa, about 161 kDa, about 162 kDa, about 163 kDa, about 164 kDa, about 165 kDa, about 166 kDa, about 167 kDa about 168 kDa, about 169 kDa, about 170, kDa about 171 kDa, about 172 kDa, about 173 kDa, about 174 kDa, about 175 kDa, about 176 kDa, about 177 kDa, about 178 kDa, about 179 kDa, about 180 kDa, about 181 kDa, about 182 kDa, about 183 kDa, about 184 kDa, about 185 kDa, about 186 kDa, about 187 kDa, about 188 kDa, about 189 kDa, about kDa 190 kDa, about 191 kDa, about 192 kDa, about 193 kDa, about 194 kDa, about 195 kDa, about 196 kDa, about 197 kDa, about 198 kDa, about 199 kDa, to about 200 kDa, including all ranges there between. In some embodiments, the polymer has a Mw of between about 5 kDa and 50 kDa. In some embodiments, the polymer has a Mw of between about 10 kDa and 50 kDa.

In some embodiments, the polymers of the present disclosure have a Mw about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, about 80 kDa, about 81 kDa, about 82 kDa, about 83 kDa, about 84 kDa, about 85 kDa, about 86 kDa, about 87 kDa, about 88 kDa, about 89 kDa, about 90 kDa, about 91 kDa, about 92 kDa, about 93 kDa, about 94 kDa, about 95 kDa, about 96 kDa, about 97 kDa, about 98 kDa, about 99 kDa, about 100 kDa about, 101 kDa, about 102 kDa, about 103 kDa, about 104 kDa, about 105 kDa, about 106 kDa, about 107 kDa, about 108 kDa, about 109 kDa, about 110 kDa, about 111 kDa, about 112 kDa, about 113 kDa, about 114 kDa, about 115 kDa, about 116 kDa, about 117 kDa, about 118 kDa, about 119 kDa, about 120 kDa, about 121 kDa, about 122 kDa, about 123 kDa, about 124 kDa, about 125 kDa, about 126 kDa, about 127 kDa, about 128 kDa, about 129 kDa, about 130 kDa, about 131 kDa, about 132 kDa, about 133 kDa, about 134 kDa, about 135 kDa, about 136 kDa, about 137 kDa, about 138, kDa about 139 kDa, about 140 kDa, about 141 kDa, about 142 kDa, about 143 kDa, about 144 kDa, about 145 kDa, about 146 kDa, about 147 kDa, about 148 kDa, about 149 kDa, about 150 kDa, about 151 kDa, about 152 kDa, about 153 kDa, about 154 kDa, about 155 kDa, about 156 kDa, about 157 kDa, about 158 kDa, about 159 kDa, about 160 kDa, about 161 kDa, about 162 kDa, about 163 kDa, about 164 kDa, about 165 kDa, about 166 kDa, about 167 kDa about 168 kDa, about 169 kDa, about 170 kDa, about 171 kDa, about 172 kDa, about 173 kDa, about 174 kDa, about 175 kDa, about 176 kDa, about 177 kDa, about 178 kDa, about 179 kDa, about 180 kDa, about 181 kDa, about 182 kDa, about 183 kDa, about 184 kDa, about 185 kDa, about 186 kDa, about 187 kDa, about 188 kDa, about 189 kDa, about 190 kDa, about 191 kDa, about 192 kDa, about 193 kDa, about 194 kDa, about 195 kDa, about 196 kDa, about 197 kDa, about 198 kDa, about 199 kDa, to about 200 kDa. In some embodiments, the polymer has a Mw of between about 5 kDa and 50 kDa. In some embodiments, the polymer has a Mw of between about 10 kDa and 50 kDa. In some embodiments, the polymer has a Mw of about 10 kDa. In some embodiments, the polymer has a Mw of about 20 kDa. In some embodiments, the polymer has a Mw of about 30 kDa. In some embodiments, the polymer has a Mw of about 40 kDa.

In some embodiments, the polymers of the present disclosure have a Mw of at least 3 kDa. In some embodiments, the polymers of the present disclosure have a Mw of between about 5 kDa and 50 kDa. In some embodiments, the polymers of the present disclosure have a Mw of about 10 kDa.

In some embodiments, the polymer and nucleic acid component are mixed in a ratio of from about 0.1:1 to about 200:1 (w/w). For example, the polymer and nucleic acid component are mixed in a ratio ranging from about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 to about 200:1, including all ranges there between. In some embodiments, the polymer and the nucleic acid component are mixed in a ratio of from about 20:1 to about 80:1 (w/w).

In some embodiments, the polymer and nucleic acid component are mixed in a ratio of about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 or about 200:1. In some embodiments, the polymer and the nucleic acid component are mixed in a ratio of about 30:1 (w/w).

In some further embodiments, the polymer has a Mw of about 3 kDa to about 200 kDa. In some further embodiments, the polymer has a Mw of about 5 kDa to about 50 kDa. In some further embodiments, the polymer has a Mw of between about 10 kDa and 50 kDa. In some further embodiments, the polymer has a Mw of about 5 kDa to about 15 kDa. In some further embodiments, the polymer has a Mw of about 10 kDa. In some further embodiments, the polymer has a Mw of about 20 kDa. In some further embodiments, the polymer has a Mw of about 30 kDa. In some further embodiments, the polymer has a Mw of about 40 kDa. In some further embodiments, the polymer has an alpha parameter defined from the Mark-Houwink equation of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In one aspect, the polymer is an HPAE.

In some embodiments, the polymer and nucleic acid component are present at a ratio of from about 0.1:1 to about 200:1 (w/w) in the polyplex. For example, the polymer and nucleic acid component are present at a ratio ranging from about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 to about 200:1 in the polyplex, including all ranges there between. In some embodiments, the polymer and nucleic acid component are present at a ratio of from about 20:1 to about 80:1 (w/w) in the polyplex.

In some embodiments, the polymer and nucleic acid component are present at a ratio of about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 or about 200:1 in the polyplex. In some embodiments, the polymer and nucleic acid component are present at a ratio of about 30:1 (w/w) in the polyplex.

In some embodiments, the particle size of the stabilized polyplex is less than 2 μm. In some embodiments, the particle size of the stabilized polyplex is less than about 300 nm. In some embodiments, the particle size of the stabilized polyplex is about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, about 150 nm, about 151 nm, about 152 nm, about 153 nm, about 154 nm, about 155 nm, about 156 nm, about 157 nm, about 158 nm, about 159 nm, about 160 nm, about 161 nm, about 162 nm, about 163 nm, about 164 nm, about 165 nm, about 166 nm, about 167 nm, about 168 nm, about 169 nm, about 170 nm, about 171 nm, about 172 nm, about 173 nm, about 174 nm, about 175 nm, about 176 nm, about 177 nm, about 178 nm, about 179 nm, about 180 nm, about 181 nm, about 182 nm, about 183 nm, about 184 nm, about 185 nm, about 186 nm, about 187 nm, about 188 nm, about 189 nm, about 190 nm, about 191 nm, about 192 nm, about 193 nm, about 194 nm, about 195 nm, about 196 nm, about 197 nm, about 198 nm, about 199 nm, about 200 nm, about 201 nm, about 202 nm, about 203 nm, about 204 nm, about 205 nm, about 206 nm, about 207 nm, about 208 nm, about 209 nm, about 210 nm, about 211 nm, about 212 nm, about 213 nm, about 214 nm, about 215 nm, about 216 nm, about 217 nm, about 218 nm, about 219 nm, about 220 nm, about 221 nm, about 222 nm, about 223 nm, about 224 nm, about 225 nm, about 226 nm, about 227 nm, about 228 nm, about 229 nm, about 230 nm, about 231 nm, about 232 nm, about 233 nm, about 234 nm, about 235 nm, about 236 nm, about 237 nm, about 238 nm, about 239 nm, about 240 nm, about 241 nm, about 242 nm, about 243 nm, about 244 nm, about 245 nm, about 246 nm, about 247 nm, about 248 nm, about 249 nm, about 250 nm, about 251 nm, about 252 nm, about 253 nm, about 254 nm, about 255 nm, about 256 nm, about 257 nm, about 258 nm, about 259 nm, about 260 nm, about 261 nm, about 262 nm, about 263 nm, about 264 nm, about 265 nm, about 266 nm, about 267 nm, about 268 nm, about 269 nm, about 270 nm, about 271 nm, about 272 nm, about 273 nm, about 274 nm, about 275 nm, about 276 nm, about 277 nm, about 278 nm, about 279 nm, about 280 nm, about 281 nm, about 282 nm, about 283 nm, about 284 nm, about 285 nm, about 286 nm, about 287 nm, about 288 nm, about 289 nm, about 290 nm, about 291 nm, about 292 nm, about 293 nm, about 294 nm, about 295 nm, about 296 nm, about 297 nm, about 298 nm, about 299 nm, or about 300 nm. In some embodiments, the stabilized polyplexes of the present disclosure have a particle size of about 60 nm to about 250 nm. In some embodiments, the stabilized polyplexes of the present disclosure have a particle size of about 175 nm to about 250 nm.

In some embodiments, the stabilized polyplex is slightly positively charged. In some embodiments, slightly positively charged is a zeta potential from about 10 and 40 mV.

In some embodiments, the stabilized polyplexes of the present disclosure have a zeta potential from about 0 mV to about 100 mV, including about 0 mV, about 1 mV, about 2 mV, about 3 mV, about 4 mV, about 5 mV, about 6 mV, about 7 mV, about 8 mV, about 9 mV, about 10 mV, about 11 mV, about 12 mV, about 13 mV, about 14 mV, about 15 mV, about 16 mV, about 17 mV, about 18 mV, about 19 mV, about 20 mV, about 21 mV, about 22 mV, about 23 mV, about 24 mV, about 25 mV, about 26 mV, about 27 mV, about 28 mV, about 29 mV, about 30 mV, about 31 mV, about 32 mV, about 33 mV, about 34 mV, about 35 mV, about 36 mV, about 37 mV, about 38 mV, about 39 mV, about 40 mV, about 41 mV, about 42 mV, about 43 mV, about 44 mV, about 45 mV, about 46 mV, about 47 mV, about 48 mV, about 49 mV, about 50 mV, about 51 mV, about 52 mV, about 53 mV, about 54 mV, about 55 mV, about 56 mV, about 57 mV, about 58 mV, about 59 mV, about 60 mV, about 61 mV, about 62 mV, about 63 mV, about 64 mV, about 65 mV, about 66 mV, about 67 mV, about 68 mV, about 69 mV, about 70 mV, about 71 mV, about 72 mV, about 73 mV, about 74 mV, about 75 mV, about 76 mV, about 77 mV, about 78 mV, about 78 mV, about 79 mV, about 80 mV, about 81 mV, about 82 mV, about 83 mV, about 84 mV, about 85 mV, about 86 mV, about 87 mV, about 88 mV, about 89 mV, about 90 mV, about 91 mV, about 92 mV, about 93 mV, about 94 mV, about 95 mV, about 96 mV, about 97 mV, about 98 mV, about 99 mV, and about 100 mV, including all ranges there between. In some embodiments, the zeta potential of the stabilized polyplex is from about 30 mV to about 34 mV.

In some embodiments, the stabilized polyplexes of the present disclosure have a zeta potential of about 0 mV, about 1 mV, about 2 mV, about 3 mV, about 4 mV, about 5 mV, about 6 mV, about 7 mV, about 8 mV, about 9 mV, about 10 mV, about 11 mV, about 12 mV, about 13 mV, about 14 mV, about 15 mV, about 16 mV, about 17 mV, about 18 mV, about 19 mV, about 20 mV, about 21 mV, about 22 mV, about 23 mV, about 24 mV, about 25 mV, about 26 mV, about 27 mV, about 28 mV, about 29 mV, about 30 mV, about 31 mV, about 32 mV, about 33 mV, about 34 mV, about 35 mV, about 36 mV, about 37 mV, about 38 mV, about 39 mV, about 40 mV, about 41 mV, about 42 mV, about 43 mV, about 44 mV, about 45 mV, about 46 mV, about 47 mV, about 48 mV, about 49 mV, about 50 mV, about 51 mV, about 52 mV, about 53 mV, about 54 mV, about 55 mV, about 56 mV, about 57 mV, about 58 mV, about 59 mV, about 60 mV, about 61 mV, about 62 mV, about 63 mV, about 64 mV, about 65 mV, about 66 mV, about 67 mV, about 68 mV, about 69 mV, about 70 mV, about 71 mV, about 72 mV, about 73 mV, about 74 mV, about 75 mV, about 76 mV, about 77 mV, about 78 mV, about 78 mV, about 79 mV, about 80 mV, about 81 mV, about 82 mV, about 83 mV, about 84 mV, about 85 mV, about 86 mV, about 87 mV, about 88 mV, about 89 mV, about 90 mV, about 91 mV, about 92 mV, about 93 mV, about 94 mV, about 95 mV, about 96 mV, about 97 mV, about 98 mV, about 99 or about 100 mV.

In some embodiments, the stabilized polyplex is spherical.

In some embodiments, the nucleic acid component of the polyplex is a plasmid, nanoplasmid, nucleic acid, minicircle, or gene editing system. In some embodiments, the nucleic acid component of the polyplex is a plasmid. In some embodiments, the nucleic acid component of the polyplex is a nanoplasmid. In some embodiments, the nanoplasmid comprises a eukaryotic transgene and a bacterial backbone that is less than 0.5 kb in size. In some embodiments, the plasmid or nanoplasmid is an antibiotic resistance marker-free plasmid or antibiotic resistance marker-free nanoplasmid. In some embodiments, the plasmid or nanoplasmid comprises a sucrose selection marker or nonsense suppressor marker.

In some embodiments, the nucleic acid component of the polyplex is a gene editing system. In some embodiments, the gene editing system is a (i) clustered, regularly interspaced, palindromic repeats (CRISPR)-associated (Cas) system; (ii) a transcription activator-like effector nuclease (TALEN) system; or (iii) a zinc finger nuclease (ZFN) system.

In some embodiments, the nucleic acid component is an RNAi-inducing molecule. The RNAi-inducing molecule may be selected from the group consisting of siRNA, dsRNA, shRNA, and microRNA.

In some embodiments, the nucleic acid component comprises a tissue-specific promoter.

In some embodiments, the nucleic acid component comprises a gene associated with a genetic disease or disorder. The genetic disease or disorder may be caused by a mutation in one or more genes that results in low, absent, or dysfunctional protein expression. In some embodiments, the gene is selected from the group consisting of COL7A1, LAMB3, ADA, SERPINA1, CFTR, HTT, NF1, PHA, HBS, FERMT1, KRT14, DSP, SPINK5, and FLG.

In some embodiments, the gene is COL7A1 and the genetic disease or disorder is a form of epidermolysis bullosa. Epidermolysis bullosa includes Epidermolysis bullosa dystrophica (autosomal recessive), Epidermolysis bullosa dystrophica (localisata variant), Epidermolysis bullosa pruriginosa, Epidermolysis bullosa (pretibial), Epidermolysis bullosa simplex (Dowling-Meara-type), Epidermolysis bullosa simplex (Koebner-type), Epidermolysis bullosa simplex (recessive 1), Epidermolysis bullosa simplex (Weber-Cockayne-type), Epidermolysis bullosa (lethal acantholytic). In some embodiments, the genetic disorder or genetic disease is adenosine deaminase (ADA) deficiency, Alpha-1 Antitrypsin Deficiency, cystic fibrosis, Huntington's Disease, Neurofibromatosis Type 1, Phenylketonuria, Sickle Cell Disease, Sporadic Inclusion Body Myositis, Duchenne muscular dystrophy, Kindler syndrome, Junctional Epidermolysis Bullosa, Dermatopathia pigmentosa reticularis, Naegeli-Franceschetti-Jadassohn syndrome, Netherton Syndrome, Ichthyosis Vulgaris, Atopic Dermatitis, Usher's syndrome, Ehlers-Danlos syndrome, Homozygous Familial Hypercholesterolemia (HoFH), or Crohn's disease.

In some embodiments, the sequence of the gene is optimized for maximum protein expression upon delivery of the polyplex to a cell.

### Pharmaceutical Compositions

In some embodiments, the present disclosure provides a pharmaceutical composition comprising an effective amount of one or more polyplexes described herein, in combination with a cryoprotectant. In some embodiments, the cryoprotectant is selected from the group consisting of glucose, sucrose, trehalose, lactose, mannitol, sorbitol, aerosil (colloidal silicon dioxide), maltose, poly(vinyl pyrrolidone), fructose, dextran, glycerol, poly(vinyl alcohol), glycine, hydroxypropyl-β-cyclodextrin, and gelatin. In some embodiments, the cryoprotectant is selected from the group consisting of trehalose, sucrose, glucose and mannitol. In some embodiments, the cryoprotectant is sucrose.

In some embodiments, the pharmaceutical composition is a lyophil. In some embodiments, the lyophil comprises an effective amount of one or more polyplexes described herein, in combination with a pharmaceutically acceptable excipient. In some embodiments, the pharmaceutically acceptable excipient comprises a cryoprotectant. In some embodiments, the cryoprotectant is selected from the group consisting of trehalose, sucrose, glucose and mannitol. In some embodiments, the cryoprotectant is sucrose.

In some embodiments, the method comprises combining one or more polyplexes described herein with a suitable solvent. In some embodiments, the suitable solvent is selected from the group consisting of water, dimethylsulfoxide and mixtures thereof. In some embodiments, the suitable solvent comprises water.

In some embodiments, the method comprises:
(a) combining one or more polyplexes described herein with a suitable solvent;
(b) adding one or more pharmaceutically acceptable excipients to the mixture of Step (a) and
(c) lyophilizing the mixture of Step (b) to provide a lyophil.

In some embodiments, the one or more pharmaceutically acceptable excipient of step (b) comprises a cryoprotectant. In some embodiments, the concentration of the cryoprotectant is from about 1% to about 20%, including about 2%, about 3%, about 4%, about 5%, about 6%, about 7%, about 8%, about 9%, about 10%, about 11%, about 12%, about 13%, about 14%, about 15%, about 16%, about 17%, about 18%, and about 19%, including all ranges therebetween, by weight of the Step (b) mixture. In some embodiments, the concentration of the cryoprotectant is about 1% about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% by weight of the Step (b) mixture. In some embodiments, the concentration of the cryoprotectant is about 1% by weight of the Step (b) mixture. In some embodiments, the concentration of the cryoprotectant is about 3% by weight of the Step (b) mixture. In some embodiments, the concentration of the cryoprotectant is about 5% by weight of the Step (b) mixture.

### EXAMPLES

The following examples are provided to illustrate the present disclosure, and should not be construed as limiting thereof. In these examples, all parts and percentages are by weight, unless otherwise noted. Abbreviations in the examples are noted below.

### Example 1

Synthesis of exemplary hyperbranched PAE (HPAE) polymer: 0.28 g trimethylolpropane triacrylate (TMPTA), 0.29 g bisphenol ethoxylate diacrylate (BE) and 0.18 g 4-amino-1-butanol (S4) are dissolved in 7.5 mL of DMSO, and reacted at 90° C. Once the molecular weight is in the range of 5000-7000 Da, 0.288 g 3-morpholinopropylamine (MPA) dissolved in 2.88 mL of DMSO is added to end-cap the acrylate terminated base polymer at RT for 24 h. The polymer product is precipitated into diethyl ether three times, dried under vacuum for 24 h and then stored at −20° C. for subsequent studies.

Polyplex preparation: the HPAE polymer is first dissolved in DMSO to a concentration of about 100 μg/μL. According to the HPAE polymer/DNA weight ratio of 30:1 (w/w), the required amount of HPAE polymer stock solution and 4 mg/mL nanoplasmid COL7A1 solution is diluted with sodium acetate buffer (0.025 M, pH=5.2) to the required volume. 10 μL of HPAE solution and 10 μL DNA solution are then dispensed through separate nozzles into a mixing chamber, where the two solutions are allowed to contact and mix. The newly formed polyplex drop flows into a slightly negatively charged liquid channel containing silicon oil/sodium acetate buffer. The polyplexes are conveyed through the liquid channel for a residence time of approximately 10 minutes at a velocity in the range of 0.1 to 1 m/s. The polyplexes with size and charge that is suitable for transdermal administration are frozen and lyophilized to be re-suspended for therapeutic use.

### Embodiments

1. A method for making one or more polyplexes, the method comprising:
   (a) providing:
      i. a polymer in a first liquid stream;
      ii. a nucleic acid component in a second liquid stream;
   (b) contacting the polymer in the first liquid stream with the nucleic acid component in the second liquid stream to form a polyplex having a size and charge that is suitable for therapeutic administration; and
   (c) isolating the polyplex to provide a stabilized polyplex.
2. The method of embodiment 1, wherein the isolating comprises conveying the polyplex in a liquid channel for a residence time sufficient to stabilize the polyplex.
3. The method of any one of the preceding embodiments, further comprising assessing and harvesting one or more polyplexes.
4. The method of any one of the preceding embodiments, wherein the first liquid stream and first liquid streams are solutions.
5. The method of any one of the preceding embodiments, further comprising flowing the first liquid stream and the second liquid stream through a flow-regulating device at a rate that provides a polyplex having a size and charge that is suitable for transdermal administration.
6. The method of embodiment 5, wherein flow-regulating device is selected from the group consisting of a positive displacement pump, a syringe driven pump, a pressure driven pump, and a gravity feed pump.
7. The method of any one of the preceding embodiments, wherein the contacting step uses a nozzle, a micro fluidics mixing device, a touch tube, a liquid bridge, a vertical mixer, a rotating double tube, or an atomizer.
8. The method any one of the preceding embodiments, wherein the contacting step occurs in an air gap or in carrier fluid within the liquid channel
9. The method of any one of the preceding embodiments, wherein the stabilized polyplex is slightly positively charged.
10. The method of any one of embodiments 2-9, wherein the liquid channel is slightly negatively charged.
11. The method of embodiment 10, wherein the liquid channel comprises an aqueous phase surrounded by a carrier fluid.
12. The method of embodiment 11, wherein the density of the carrier fluid is a value in the range of 1,300 to 2,000 kg/m³ and the density of the aqueous phase is a value in the range of 900 to 1200 kg/m³.
13. The method of embodiment 12, wherein the aqueous phase comprises sodium acetate buffer.
14. The method of any embodiment 11, wherein the carrier fluid is an oil.
15. The method of embodiment 14, wherein the oil is selected from the group consisting of: (a) Fluorinert FC-40 (fluorocarbonated oil), (b) silicon oil, (c) mineral oil, (d) perfluorinated amine oil, (e) phenylmethylpolysiloxane and (f) phenylmethylpolysiloxane-based oil and a additive.
16. The method of embodiment 15, wherein the additive has a hydrophilic-lipophilic balance number in the range of 2 to 8.
17. The method of embodiment 15, wherein the additive is a polysorbate additive.
18. The method of embodiment 17, wherein the polysorbate additive is SPAN 80, SPAN 65 or Tween 20.
19. The method of embodiment 15, wherein the concentration of the additive in the aqueous phase is from about 0.001% and about 10% (wt/wt %).
20. The method of any one of any one of the preceding embodiments, wherein the isolating comprises segregating the polyplexes in the liquid channel
21. The method of embodiment 20, wherein the space between the segregated polyplexes is controlled by the velocity of liquid in the channel
22. The method of any one of embodiments 2-21, wherein the residence time is from about 1 second to about 20 minutes.
23. The method of embodiment 22, wherein the residence time is about 10 minutes.
24. The method of any one of embodiments 3-23, wherein assessing comprises: measuring the refractive index of polyplexes in the liquid channel; and optionally removing polyplexes with a refractive index which does not conform to specification.
25. The method of any one of the preceding embodiments, further comprising filtering, washing, freezing and/or lyophilizing the stabilized polyplexes.
26. The method of any one of the preceding embodiments, wherein the polymer is positively charged and the nucleic acid component is negatively charged.
27. The method of any one of the preceding embodiments, wherein the polymer and nucleic acid component are mixed in a ratio of from about 0.1:1 to about 200:1 (w/w).
28. The method of embodiment 27, wherein the polymer and the nucleic acid component are mixed in a ratio of from about 20:1 to about 80:1 (w/w).
29. The method of embodiment 27, wherein the polymer and the nucleic acid component are mixed in a ratio of about 30:1 (w/w).
30. The method of any one of the preceding embodiments, wherein the polymer is an HPAE.
31. The method of any one of the preceding embodiments, wherein the polymer and nucleic acid component are present at a ratio of from about 0.1:1 to about 200:1 (w/w) in the polyplex.
32. The method of embodiment 31, wherein polymer and nucleic acid component are present at a ratio of from about 20:1 to about 80:1 (w/w) in the polyplex.
33. The method of embodiment 31, wherein polymer and nucleic acid component are present at a ratio of about 30:1 (w/w) in the polyplex.
34. The method of any one of the preceding embodiments, wherein the particle size of the stabilized polyplex is less than about 2 μm.
35. The method of embodiment 34, wherein the particle size of the stabilized polyplex is about 60 nm to about 250 nm.
36. The method of embodiment 34, wherein the particle size of the stabilized polyplex is about 175 nm to about 250 nm.
37. The method of any one of the preceding embodiments, wherein the zeta potential of the stabilized polyplex is from about 0 mV to about 100 mV.
38. The method of embodiment 37, wherein the zeta potential of the stabilized polyplex is from about 30 mV to about 34 mV.
39. The method of any one of the preceding embodiments, wherein the stabilized polyplex is spherical.
40. The method of any one of the preceding embodiments, wherein the polymer has a Mw of about 10 kDa.
41. The method of any one of the preceding embodiments, wherein the nucleic acid component is a plasmid, nanoplasmid, nucleic acid, minicircle, or gene editing system.
42. The method of embodiment 41, wherein the nucleic acid component comprises a gene associated with a genetic disease or disorder.
43. The method of embodiment 42, wherein the genetic disease or disorder is caused by a mutation in one or more genes that results in low, absent, or dysfunctional protein expression.
44. The method of embodiment 43, wherein the gene is selected from the group consisting of COL7A1, LAMB3, ADA, SERPINA1, CFTR, HTT, NF1, PHA, HBS, FERMT1, KRT14, DSP, SPINK5, and FLG.
45. The method of embodiment 44, wherein the gene is COL7A1 and the genetic disease or disorder is a form of epidermolysis bullosa.
46. The method of embodiment 45, wherein the sequence of the gene is optimized for maximum protein expression upon delivery of the polyplex to a cell.
47. The method of any one of the preceding embodiments, wherein the polymer has an alpha parameter defined from the Mark-Houwink equation of less than about 0.5.
48. The method of any one of the preceding embodiments, wherein the polymer has an alpha parameter defined from the Mark-Houwink equation from about 0.2 to about 0.5.
49. The method of any one of the preceding embodiments, wherein the polymer has a PDI from about 1.01 to about 8.0.
50. The method of any one of the preceding embodiments, wherein the polymer has a PDI of about 2.5.
51. The method of any one of the preceding embodiments, wherein the polymer has a Mw of at least 3 kDa.
52. The method of any one of the preceding embodiments, wherein the polymer has a Mw of between about 5 kDa and 50 kDa.
53. The method of any one of the preceding embodiments, wherein the polymer has a Mw of about 10 kDa.
54. The method of any one of the preceding embodiments, wherein the polymer comprises:

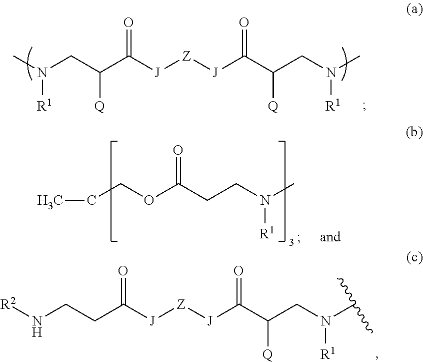

wherein
J is O and Z is

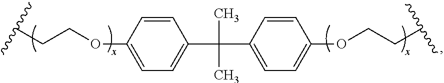

wherein x is 1-1000;
R₁ is

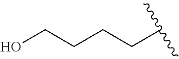 [SMILES: *CCCCO]

and

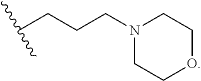 [SMILES: *CCCN1CCOCC1]

R₂ is

### REFERENCES

[1] L. Naldini, Gene therapy returns to centre stage. Nature 2015.
[2] H. Yin, R. L. Kanasty, A. A. Eltoukhy, A. J. Vegas, J. R. Dorkin, D. G. Anderson, Non-viral vectors for gene-based therapy. Nat. Rev. Genet. 2014.
[3] M. Foldvari, D. W. Chen, N. Nafissi, D. Calderon, L. Narsineni, A. Rafiee, J. Control. Release 2016.
[4] H. Lv, S. Zhang, B. Wang, S. Cui, J. Yan, Toxicity of cationic lipids and cationic polymers in gene delivery. J. Control. Release 2006.
[5] C. Tros de Ilarduya, Y. Sun, N. Düzgüneş, Gene delivery by lipoplexes and polyplexes. Eur. J. Pharm. Sci. 2010, pages 159-170.

What is claimed:

1. A method for making one or more polyplexes, the method comprising:
(a) providing:
i. a polymer in a first liquid stream;
ii. a nucleic acid component in a second liquid stream;
(b) contacting the polymer in the first liquid stream with the nucleic acid component in the second liquid stream to form a polyplex having a size and charge that is suitable for therapeutic administration; and
(c) isolating the polyplex to provide a stabilized polyplex.

2. The method of claim 1, wherein the isolating comprises conveying the polyplex in a liquid channel for a residence time sufficient to stabilize the polyplex.

3. The method of any one of the preceding claims, further comprising assessing and harvesting one or more polyplexes.

4. The method of any one of the preceding claims, wherein the first liquid stream and first liquid streams are solutions.

5. The method of any one of the preceding claims, further comprising flowing the first liquid stream and the second liquid stream through a flow-regulating device at a rate that provides a polyplex having a size and charge that is suitable for transdermal administration.

6. The method of claim 5, wherein flow-regulating device is selected from the group consisting of a positive displacement pump, a syringe driven pump, a pressure driven pump, and a gravity feed pump.

7. The method of any one of the preceding claims, wherein the contacting step uses a nozzle, a micro fluidics mixing device, a touch tube, a liquid bridge, a vertical mixer, a rotating double tube, or an atomizer.

8. The method any one of the preceding claims, wherein the contacting step occurs in an air gap or in carrier fluid within the liquid channel.

9. The method of any one of the preceding claims, wherein the stabilized polyplex is slightly positively charged.

10. The method of any one of claims 2-9, wherein the liquid channel is slightly negatively charged.

11. The method of claim 10, wherein the liquid channel comprises an aqueous phase surrounded by a carrier fluid.

12. The method of claim 11, wherein the density of the carrier fluid is a value in the range of 1,300 to 2,000 kg/m³ and the density of the aqueous phase is a value in the range of 900 to 1200 kg/m³.

13. The method of claim 12, wherein the aqueous phase comprises sodium acetate buffer.

14. The method of any claim 11, wherein the carrier fluid is an oil.

15. The method of claim 14, wherein the oil is selected from the group consisting of: (a) Fluorinert FC-40 (fluorocarbonated oil), (b) silicon oil, (c) mineral oil, (d) perfluorinated amine oil, (e) phenylmethylpolysiloxane and (f) phenylmethylpolysiloxane-based oil and a additive.

16. The method of claim 15, wherein the additive has a hydrophilic-lipophilic balance number in the range of 2 to 8.

17. The method of claim 15, wherein the additive is a polysorbate additive.

18. The method of claim 17, wherein the polysorbate additive is SPAN 80, SPAN 65 or Tween 20.

19. The method of claim 15, wherein the concentration of the additive in the aqueous phase is from about 0.001% and about 10% (wt/wt %).

20. The method of any one of any one of the preceding claims, wherein the isolating comprises segregating the polyplexes in the liquid channel.

21. The method of claim 20, wherein the space between the segregated polyplexes is controlled by the velocity of liquid in the channel.

22. The method of any one of claims 2-21, wherein the residence time is from about 1 second to about 20 minutes.

23. The method of claim 22, wherein the residence time is about 10 minutes.

24. The method of any one of claims 3-23, wherein assessing comprises:
measuring the refractive index of polyplexes in the liquid channel; and
optionally removing polyplexes with a refractive index which does not conform to specification.

25. The method of any one of the preceding claims, further comprising filtering, washing, freezing and/or lyophilizing the stabilized polyplexes.

26. The method of any one of the preceding claims, wherein the polymer is positively charged and the nucleic acid component is negatively charged.

27. The method of any one of the preceding claims, wherein the polymer and nucleic acid component are mixed in a ratio of from about 0.1:1 to about 200:1 (w/w).

28. The method of claim 27, wherein the polymer and the nucleic acid component are mixed in a ratio of from about 20:1 to about 80:1 (w/w).

29. The method of claim 27, wherein the polymer and the nucleic acid component are mixed in a ratio of about 30:1 (w/w).

30. The method of any one of the preceding claims, wherein the polymer is an HPAE.

31. The method of any one of the preceding claims, wherein the polymer and nucleic acid component are present at a ratio of from about 0.1:1 to about 200:1 (w/w) in the polyplex.

32. The method of claim 31, wherein polymer and nucleic acid component are present at a ratio of from about 20:1 to about 80:1 (w/w) in the polyplex.

33. The method of claim 31, wherein polymer and nucleic acid component are present at a ratio of about 30:1 (w/w) in the polyplex.

34. The method of any one of the preceding claims, wherein the particle size of the stabilized polyplex is less than about 2 μm.

35. The method of claim 34, wherein the particle size of the stabilized polyplex is about 60 nm to about 250 nm.

36. The method of claim 34, wherein the particle size of the stabilized polyplex is about 175 nm to about 250 nm.

37. The method of any one of the preceding claims, wherein the zeta potential of the stabilized polyplex is from about 0 mV to about 100 mV.

38. The method of claim 37, wherein the zeta potential of the stabilized polyplex is from about 30 mV to about 34 mV.

39. The method of any one of the preceding claims, wherein the stabilized polyplex is spherical.

40. The method of any one of the preceding claims, wherein the polymer has a Mw of about 10 kDa.

41. The method of any one of the preceding claims, wherein the nucleic acid component is a plasmid, nanoplasmid, nucleic acid, minicircle, or gene editing system.

42. The method of claim 41, wherein the nucleic acid component comprises a gene associated with a genetic disease or disorder.

43. The method of claim 42, wherein the genetic disease or disorder is caused by a mutation in one or more genes that results in low, absent, or dysfunctional protein expression.

44. The method of claim 43, wherein the gene is selected from the group consisting of COL7A1, LAMB3, ADA, SERPINA1, CFTR, HTT, NF1, PHA, HBS, FERMT1, KRT14, DSP, SPINK5, and FLG.

45. The method of claim 44, wherein the gene is COL7A1 and the genetic disease or disorder is a form of epidermolysis bullosa.

46. The method of claim 45, wherein the sequence of the gene is optimized for maximum protein expression upon delivery of the polyplex to a cell.

47. The method of any one of the preceding claims, wherein the polymer has an alpha parameter defined from the Mark-Houwink equation of less than about 0.5.

48. The method of any one of the preceding claims, wherein the polymer has an alpha parameter defined from the Mark-Houwink equation from about 0.2 to about 0.5.

49. The method of any one of the preceding claims, wherein the polymer has a PDI from about 1.01 to about 8.0.

50. The method of any one of the preceding claims, wherein the polymer has a PDI of about 2.5.

51. The method of any one of the preceding claims, wherein the polymer has a Mw of at least 3 kDa.

52. The method of any one of the preceding claims, wherein the polymer has a Mw of between about 5 kDa and 50 kDa.

53. The method of any one of the preceding claims, wherein the polymer has a Mw of about 10 kDa.
